(19) United States
(12) Patent Application Publication
(10) Pub. No.: US 2017/0143966 A1
(43) Pub. Date: May 25, 2017

(54) SYSTEMS AND METHODS FOR DETERMINING A TRAJECTORY FOR A BRAIN STIMULATION LEAD

(71) Applicant: Functional Neuromodulation, Inc., Toronto, CA (US)

(72) Inventors: Donald E. Reymers, Brevard, NC (US); Mark Rise, Monticello, MN (US); Daniel J. O'Connell, Earlysville, VA (US); R. Maxwell Flaherty, Auburndale, FL (US); J. Christopher Flaherty, Auburndale, FL (US)

(21) Appl. No.: 15/279,308

(22) Filed: Sep. 28, 2016

Related U.S. Application Data
(60) Provisional application No. 61/972,704, filed on Mar. 31, 2014.

(51) Int. Cl. A61N 1/36; A61B 8/00; A61B 6/03; A61B 5/0476; A61B 5/021; A61M 5/142; A61B 5/00; A61N 2/00; A61N 1/05; A61N 5/06; A61N 7/00; A61B 5/055; A61B 5/145

(52) U.S. Cl. CPC A61N 1/36082; A61B 5/055; A61B 8/4245; A61B 6/032; A61B 6/037; A61B 5/0476; A61B 5/021; A61B 5/14539; A61B 5/14542; A61B 5/0008; A61N 2/006; A61N 1/0534; A61N 5/0622; A61N 5/0601; A61N 7/00; A61M 5/14276; A61N 2005/0662; A61N 2005/0661; A61N 2005/0659; A61N 2005/0612; A61N 2007/0026

(57) ABSTRACT

Systems and methods for treating a cognitive disease or disorder are provided. A treatment method comprises: selecting a target volume of brain tissue to be stimulated; identifying at least one avoidance volume of brain tissue; selecting a first stimulation lead comprising at least one stimulation element; identifying at least one proposed trajectory for placement of the first stimulation lead based on the target volume and the at least one avoidance volume; placing the first stimulation lead along a placement trajectory selected from the at least one proposed trajectory; attaching the first stimulation lead to a stimulator; and stimulating the target volume with the first stimulation lead at least one stimulation element to treat at least one of a cognitive disease or a cognitive disorder. Systems include a stimulator with one or more stimulation leads and an image analyzer for identifying a proposed trajectory for placing the stimulation leads.

### CROSS-REFERENCE TO RELATED APPLICATIONS

This application is a continuation of PCT/CA2015/050249 (Attorney Docket No. 41551-710.601), filed Mar. 31, 2015, which claims priority under 35 USC 119(3) to U.S. Provisional Application Ser. No. 61/972,704, entitled “Systems and Methods for Determining a Trajectory for a Brain Stimulation Lead”, filed Mar. 31, 2014, which are incorporated herein by reference in their entirety.

This application is related to U.S. patent application Ser. No. 14/034,336, entitled “Cognitive Function within a Human Brain”, filed Sep. 23, 2013; U.S. Pat. No. 8,612,006, entitled “Inducing Neurogenesis within a Human Brain”, filed Dec. 16, 2005; U.S. patent application Ser. No. 14/508,110, entitled “Regulation of Neurotrophins”, filed Oct. 7, 2014; U.S. patent application Ser. No. 14/586,849, entitled “Method of Treating Cognitive Disorders Using Neuromodulation”, filed Dec. 30, 2014; U.S. patent application Ser. No. 13/655,652, entitled “Deep Brain Stimulation of Memory Circuits in Alzheimer's Disease”, filed Oct. 19, 2012; and International PCT Application Serial Number PCT/US2014/060923, entitled “Brain Stimulation System including Diagnostic Tool”, filed Oct. 16, 2014; the contents of which are each incorporated herein by reference in their entirety.

### FIELD OF INVENTION

The present invention relates generally to methods and systems for treating a neurological disease or disorder, such as Alzheimer's Disease or other cognitive disorder. In particular, a system includes a stimulation device, a stimulation lead and an image analyzer used to determine a trajectory for placement of the stimulation lead.

### BACKGROUND OF THE INVENTION

Brain stimulation has been performed to treat numerous patient diseases and disorders, such as neurological and psychiatric conditions. Both invasive and non-invasive technologies have been developed. One non-invasive system includes a transcranial magnetic stimulation device that directs a magnetic field from outside the patient's head to induce electric currents in the patient's brain. Deep brain stimulation (DBS) can be accomplished using surgically implanted electrodes that deliver electrical stimulation to precisely targeted areas in the brain. More than 60,000 patients have been implanted with deep brain electrodes, and its predominant application has been in the treatment of movement disorders, most commonly Parkinson's disease.

There is a need for enhanced DBS and other brain stimulation systems, device and methods that result in increased safety and improved efficacy in the treatment of patients.

### BRIEF SUMMARY OF THE INVENTION

According to an aspect of the present inventive concepts, a method of treating a cognitive disease or disorder comprises: selecting a target volume of brain tissue to be stimulated; identifying at least one avoidance volume of brain tissue; selecting a first stimulation lead comprising at least one stimulation element; identifying at least one proposed trajectory for placement of the first stimulation lead based on the target volume and the at least one avoidance volume; placing the first stimulation lead along a placement trajectory selected from the at least one proposed trajectory; attaching the first stimulation lead to a stimulator; and stimulating the target volume with the first stimulation lead at least one stimulation element to treat at least one of a cognitive disease or a cognitive disorder.

In some embodiments, the cognitive disease or disorder comprises a disease or disorder selected from the group consisting of: Alzheimer's Disease (AD) such as Mild or Moderate Alzheimer's Disease; probable Alzheimer's Disease; a genetic form of Alzheimer's Disease; Mild Cognitive Impairment (MCI); hippocampal damage such as hippocampal damage due to Alzheimer's disease, anoxia, epilepsy or depression; neuronal loss; neuronal damage; chemotherapy induced memory impairment; epilepsy; a seizure disorder; dementia; amnesia; a memory disorder such a spatial memory disorder; traumatic brain injury; cognitive impairment associated with Schizophrenia; Parkinson's Disease related cognitive impairment or dementia; and combinations thereof.

In some embodiments, the method is configured to treat negative symptoms of a disease or disorder selected from the group consisting of: schizophrenia; depression; other conditions of reversible impaired memory or cognition; and combinations thereof.

In some embodiments, the target volume comprises at least a portion of the fornix. The target volume can further comprise non-fornix brain tissue.

In some embodiments, the target volume comprises brain tissue selected from the group consisting of: fornix; entorhinal cortex; hippocampus; anterior thalamic nucleus; amygdala; mammilary bodies; parahippocampal cortex; temporal neocortex; septal nuclei; nucleus basalis of Meynert; subcallosal or subgenual cingulate; ventral capsule; ventral striatum; anterior commissure; corpus callosum; and combinations thereof.

In some embodiments, the target volume comprises brain tissue selected from the group consisting of: Papez Circuit; hippocampus; cingulate gyrus; fornix; a mammilothalamic tract; amygdala; hypothalamus; mammilary bodies; septal nuclei; temporal neocortex; the medial forebrain bundle; anterior and mediodorsal nuclei of the thalamus; the diagonal band of the Broca; temporal stem and temporal white matter; brainstem; nucleus basalis of Meynert; anterior thalamic nucleus; entorhinal cortex; rhinal cortex; periventricular zone; anterior thalamus; anterior insula; caudate; dorsal anterior cortex; dorsal cingulate; medial frontal cortex; nucleus accumbens; orbital frontal cortex; parietal region; periaqueductal gray area; posterior cingulate area; subcallosal area; subcallosal cingulate; subgenual cingulate; Brodmann area 10; Brodmann area 24; Brodmann area 25; Brodmann area 11/Brodmann area 10; Brodmann area 24b; Brodmann area 31; Brodmann area 32/Brodmann area 10; Brodmann area 32/Brodmann area 11; Brodmann area 39; Brodmann area 46; Brodmann area 46/Brodmann area 9; Brodmann area 47; Brodmann area 6; Brodmann area 9; ventral/medial prefrontal cortex area; ventral/medial white matter; dorsolateral prefrontal cortex; premotor cortex; ventrolateral prefrontal cortex; dorsal anterior cingulate caudate nucleus; frontal pole periaqueductal gray area; dorsolateral prefrontal area; subsingular cingulate; parahippocampal cortex; parahippocampal gyrus; ventral capsule; ventral striatum; and combinations thereof.

In some embodiments, the at least one avoidance volume is identified to prevent the at least one stimulation lead from passing through the avoidance volume. The at least one avoidance volume can be identified to prevent the at least one stimulation lead from damaging the avoidance volume. The at least one avoidance volume can be identified to prevent the at least one stimulation lead from deflecting off of the avoidance volume.

In some embodiments, the at least one avoidance volume is identified to minimize stimulation by the at least one stimulation element.

In some embodiments, the at least one avoidance volume of brain tissue comprises at least one blood vessel. The at least one blood vessel comprises a blood vessel selected from the group consisting of: an artery of the brain; a vein of the brain; and combinations thereof.

In some embodiments, the at least one avoidance volume of brain tissue comprises tissue proximate to a sulcus.

In some embodiments, the at least one avoidance volume of brain tissue comprises tissue proximate to a ventricular wall of the brain.

In some embodiments, the at least one avoidance volume of brain tissue comprises optical tract tissue.

In some embodiments, the at least one avoidance volume of brain tissue comprises a volume of tissue which when stimulated activates at least one of an autonomic or vegetative physiological response detrimental to the health of the patient.

In some embodiments, the at least one avoidance volume of brain tissue comprises a volume of tissue which when stimulated activates a sensory response unpleasant for the patient.

In some embodiments, the at least one avoidance volume comprises a volume of tissue selected from the group consisting of: posterior hypothalmic area; ventral tegmental area; lateral hypothalimc area; anterior hypothalamic nucleus; paraventricular nucleus; dorsal medial hypothalamic nucleus; ventromedial hypothalamic nucleus; arcuate nucleus; lateral tuberal nucleus; medial preoptic nucleus; supraoptic nucleus; and combinations thereof.

In some embodiments, the at least one avoidance volume comprises tissue of the fornix.

In some embodiments, the at least one avoidance volume comprises tissue of the hypothalamus.

In some embodiments, identifying the at least one avoidance volume of brain tissue comprises performing an imaging procedure to produce one or more images and assessing the one or more images. Identifying the at least one avoidance volume of brain tissue can comprise a manual assessment of the one or more images, such as a manual assessment performed by a clinician or other operator. Identifying the at least one avoidance volume of brain tissue can comprise an image assessment performed by an image analyzer. The imaging procedure can be performed using an imaging device selected from the group consisting of: MRI; fMRI; X-ray; fluoroscope; Ct-Scanner; PET Scanner; Diffusion Tensor Imaging device; ultrasound imaging device; and combinations thereof. The imaging procedure can comprise collecting images using T1 weighted MRI. The imaging procedure can comprise performing a contrast injection and collecting images after the contrast injection.

In some embodiments, the at least one proposed trajectory is identified to position the first stimulation lead at least one stimulation element proximate the target volume. The at least one proposed trajectory can be identified to avoid the first stimulation lead from penetrating the target volume. The target volume can comprise the fornix. The at least one proposed trajectory can be identified to avoid the first stimulation lead from penetrating the at least one avoidance volume.

In some embodiments, the at least one proposed trajectory is identified to avoid the first stimulation lead from penetrating the at least one avoidance volume. The at least one avoidance volume can comprise tissue selected from the group consisting of: tissue proximate a sulcus; blood vessel tissue; tissue proximate ventricular wall; optical tract tissue; and combinations thereof. The at least one proposed trajectory can be identified to avoid the first stimulation lead from being positioned within a threshold distance of the at least one avoidance volume. The threshold distance can be set by a clinician. The at least one avoidance volume can comprise a first avoidance volume of brain tissue and a second avoidance volume of brain tissue, and wherein the at least one proposed trajectory is identified to avoid the first stimulation lead from being positioned within a first threshold distance of the first avoidance volume and a second threshold distance of the second avoidance volume. The first threshold distance and the second threshold distance can comprise similar distances. The first threshold distance and the second threshold distance can comprise dissimilar threshold distances. At least one of the first threshold distance or the second threshold distance can be set by a clinician.

In some embodiments, the at least one proposed trajectory is identified to avoid penetrating the target volume. The at least one proposed trajectory can be identified to avoid penetrating the target volume by a threshold distance. The target volume can comprise the fornix. The threshold distance can be set by a clinician.

In some embodiments, the at least one proposed trajectory is identified to avoid the first stimulation lead from penetrating the fornix.

In some embodiments, the at least one proposed trajectory is identified to avoid passing through the target volume. The target volume can comprise the fornix.

In some embodiments, the at least one proposed trajectory is identified to position the first stimulation lead at least one stimulation element approximately 2 mm or less from the anterior border of the fornix.

In some embodiments, the at least one proposed trajectory is identified to pass through a gyrus.

In some embodiments, the target volume comprises a major axis, and the at least one proposed trajectory is identified to be relatively parallel with the major axis of the target volume. The target volume can comprise at least a portion of the fornix.

In some embodiments, the at least one proposed trajectory is identified to comprise a lateral angle between 8° and 10° from the mid-sagittal plane.

In some embodiments, the at least one proposed trajectory is identified to comprise an anterior angle between 50° and 60° from the axial plane.

In some embodiments, the at least one proposed trajectory is identified to pass through a lateral ventricle.

In some embodiments, the at least one proposed trajectory is identified to minimize deflection of the first stimulation lead as it placed along the placement trajectory. The at least one proposed trajectory can be identified to avoid being proximate and parallel to a wall of a ventricle.

In some embodiments, the at least one proposed trajectory is identified based on avoiding stimulation of non-target tissue. The non-target tissue can comprise tissue selected from the group consisting of: hippocampal tissue; optical tract tissue; and combinations thereof. The non-target tissue can comprise tissue selected from the group consisting of: posterior hypothalmic area; ventral tegmental area; lateral hypothalimc area; anterior hypothalamic nucleus; paraventricular nucleus; dorsal medial hypothalamic nucleus; ventromedial hypothalamic nucleus; arcuate nucleus; lateral tuberal nucleus; medial preoptic nucleus; supraoptic nucleus; and combinations thereof.

In some embodiments, the at least one proposed trajectory is selected further based on reducing the distance between the first stimulation lead at least one stimulation element and the target volume.

In some embodiments, the first stimulation lead at least one stimulation element comprises multiple stimulation elements, and the at least one proposed trajectory is selected further based on reducing the distance between at least two of the multiple stimulation elements and the target volume.

In some embodiments, the first stimulation lead at least one stimulation element comprises multiple stimulation elements positioned along a first axis and wherein the target volume comprises a second axis, and the at least one proposed trajectory is selected further based on increasing parallelism between the first axis and the second axis.

In some embodiments, the at least one proposed trajectory comprises a set of multiple trajectories. The set of multiple trajectories can represent at least one relatively continuous volume of tissue. The set of multiple trajectories can represent two or more relatively continuous volumes of tissue. The at least one relatively continuous volume of tissue can comprise a triangular or trapezoidal cross-sectional geometry. The at least one relatively continuous volume of tissue can comprise multiple relatively continuous volumes of tissue each with a triangular or trapezoidal cross-sectional geometry.

In some embodiments, the first stimulation lead comprises a distal portion including a distal end, and the first stimulation lead at least one stimulation element is positioned along the distal portion. Placing of the first stimulation lead can comprise positioning the first stimulation lead distal end approximately 1 cm or less beyond a ventricle of the brain. Placing of the first stimulation lead can comprise positioning the distal end of the first stimulation lead away from the optic tract. Placing of the first stimulation lead can comprise positioning the distal end of the first stimulation lead approximately 1 mm away from the optic tract. Placing of the first stimulation lead can comprise positioning the first stimulation lead distal portion to avoid stimulating non-target tissue. The non-target tissue can comprise tissue selected from the group consisting of: hippocampal tissue; hypothalamus; mammillary body; optical tract tissue; and combinations thereof. The non-target tissue can comprise tissue selected from the group consisting of: posterior hypothalamic area; ventral tegmental area; lateral hypothalamic area; anterior hypothalamic nucleus; paraventricular nucleus; dorsal medial hypothalamic nucleus; ventromedial hypothalamic nucleus; arcuate nucleus; lateral tuberal nucleus; medial preoptic nucleus; supraoptic nucleus; and combinations thereof.

In some embodiments, the first stimulation lead at least one stimulation element comprises multiple stimulation elements, and the method further comprises selecting one or more stimulation elements of the multiple stimulation elements to stimulate the target volume.

In some embodiments, the first stimulation lead at least one stimulation element comprises a stimulation element selected from the group consisting of: an electrode such as one or more electrodes configured to deliver electrical stimulation energy; a magnetic field delivery element; a light delivery element such as a visible, ultraviolet and/or infrared light delivery element; an optogenetic delivery element; a sound delivery element such as a subsonic wave and/or ultrasound wave delivery element; an agent delivery element such as a chemical and/or pharmaceutical agent delivery element; and combinations thereof.

In some embodiments, the method further comprises: selecting a second stimulation lead comprising at least one stimulation element; and identifying at least one proposed second lead trajectory for placement of the second stimulation lead based on the target volume and the at least one avoidance volume. The method can further comprise: placing the second stimulation lead along a placement trajectory selected from the at least one proposed second lead trajectory; attaching the second stimulation lead to the stimulator; and stimulating the target volume with the second lead at least one stimulation element. The first stimulation lead at least one stimulation element and the second stimulation lead at least one stimulation element can be positioned on relative opposite sides of the target volume. The target volume can comprise the fornix. The target volume can comprise the anterior pillars of the fornix, and the first stimulation lead can be positioned to stimulate a first anterior pillar of the fornix and the second stimulation lead can be positioned to stimulate a second anterior pillar of the fornix.

According to another aspect of the present inventive concepts, a system for treating a cognitive disease or disorder comprises: a stimulator configured to provide stimulation energy; a first stimulation lead constructed and arranged to receive the stimulation energy from the stimulator and comprising at least one stimulation element constructed and arranged to stimulate a target volume of brain tissue; a controller configured to modify the stimulation energy delivered by the stimulator; an image analyzer configured to receive patient image information and identify at least one proposed trajectory for placement of the first stimulation lead. The identification of the at least one proposed trajectory is based on: the target volume of brain tissue, and an avoidance volume of brain tissue. The system can be configured to treat at least one of a neurological disease or a neurological disorder.

In some embodiments, the system further comprises an imaging device configured to produce the patient image information. The imaging device can comprise a device selected from the group consisting of: MRI; fMRI; X-ray; fluoroscope; Ct-Scanner; PET Scanner; Diffusion Tensor Imaging device; ultrasound imaging device; and combinations thereof.

In some embodiments, the system further comprises a second stimulation lead constructed and arranged to receive the stimulation energy from the stimulator and comprising at least one stimulation element constructed and arranged to stimulate a second target volume of brain tissue. The target volume of brain tissue and the second volume of brain tissue can comprise similar tissue.

In some embodiments, the stimulator is configured to provide energy selected from the group consisting of: electrical energy; magnetic field energy; light energy; optogenetic energy; sound energy; chemical energy; and combinations thereof.

In some embodiments, the first stimulation lead at least one stimulation element comprises a stimulation element selected from the group consisting of: an electrode such as one or more electrodes configured to deliver electrical stimulation energy; a magnetic field delivery element; a light delivery element such as a visible, ultraviolet and/or infrared light delivery element; an optogenetic delivery element; a sound delivery element such as a subsonic wave and/or ultrasound wave delivery element; an agent delivery element such as a chemical and/or pharmaceutical agent delivery element; and combinations thereof.

In some embodiments, the image analyzer is configured to provide a qualitative measure of a selected trajectory. The qualitative measure can comprise a qualitative measure of the at least one trajectory's avoidance of the avoidance volume. The qualitative measure can comprise a qualitative measure of one or more of: proximity of the first stimulation lead to the avoidance volume and average distance between the first stimulation lead and the avoidance volume. The qualitative measure can comprise a qualitative measure of the at least on trajectory's geometric relationship to the target volume. The qualitative measure can comprise a qualitative measure of one or more of: proximity of a stimulation element to the target volume; average proximity of an array of stimulation elements to the target volume; and degree of parallelism of an array of stimulation elements to the target volume. The image analyzer can be configured to provide a first qualitative measure of a first trajectory and a second qualitative measure of a second trajectory.

In some embodiments, the image analyzer is configured to provide a quantitative measure of a selected trajectory. The quantitative measure can comprise a quantitative measure of the at least on trajectory's avoidance of the avoidance volume. The quantitative measure can comprise a quantitative measure of one or more of: proximity of the first stimulation lead to the avoidance volume and average distance between the first stimulation lead and the avoidance volume. The quantitative measure can comprise a quantitative measure of the at least on trajectory's geometric relationship to the target volume. The quantitative measure can comprise a quantitative measure of one or more of: proximity of a stimulation element to the target volume; average proximity of an array of stimulation elements to the target volume; and degree of parallelism of an array of stimulation elements to the target volume. The image analyzer can be configured to provide a first quantitative measure of a first trajectory and a second quantitative measure of a second trajectory.

In some embodiments, the image analyzer is configured to provide information related to the distance between the first stimulation lead and the avoidance volume.

In some embodiments, the image analyzer is configured to provide information related to the distance between the first at least one stimulation element and the target volume.

In some embodiments, the image analyzer is configured to allow an operator to perform a function selected from the group consisting of: select a patient image; rotate an image; magnify a patient image; select a target volume; magnify the view of a target volume; view an avoidance volume; magnify an avoidance volume; view one or more stimulation lead trajectories identified by the image analyzer; analyze one or more stimulation lead trajectories identified by the image analyzer; select one or more stimulation lead trajectories identified by the image analyzer; measure the distance between one or more stimulation lead trajectories and a target volume; measure the distance between one or more stimulation lead trajectories and an avoidance volume; measure the distance between one or more stimulation elements proposed locations and a target volume; measure the distance between one or more stimulation elements proposed locations and an avoidance volume; average one or more measured distances; sum two or more measured distances; and combinations thereof.

In some embodiments, the image analyzer comprises the controller.

In some embodiments, the system further comprises a sensor. The sensor can comprise a sensor selected from the group consisting of: neuronal activity sensor; EEG sensor; local field potential sensor; neurochemical sensor; pH sensor; pressure sensor; blood pressure sensor; optical sensor; blood gas sensor; blood oxygen sensor; magnetic sensor; strain gauge; temperature sensor; and combinations thereof.

The technology described herein, along with the attributes and attendant advantages thereof, will best be appreciated and understood in view of the following detailed description taken in conjunction with the accompanying drawings in which representative embodiments are described by way of example.

### BRIEF DESCRIPTION OF THE DRAWINGS

The foregoing and other objects, features and advantages of embodiments of the present inventive concepts will be apparent from the more particular description of preferred embodiments, as illustrated in the accompanying drawings in which like reference characters refer to the same or like elements. The drawings are not necessarily to scale, emphasis instead being placed upon illustrating the principles of the preferred embodiments.

FIG. 1 illustrates a schematic view of a system for stimulating one or more portions of a patient's brain, consistent with the present inventive concepts.

FIG. 2 illustrates a flow chart of a method for treating a patient with a brain stimulation system, consistent with the present inventive concepts.

FIG. 3 illustrates a schematic of an electrical brain stimulator, consistent with the present inventive concepts.

FIG. 4 illustrates a schematic view of a display of an image analyzer providing multiple views of a patient's brain and multiple proposed trajectories for stimulation lead placement, consistent with the present inventive concepts.

FIG. 5 illustrates an anatomical view of a portion of a patient's brain, including multiple identified trajectories for stimulation lead placement, consistent with the present inventive concepts.

FIG. 6 illustrates lateral and frontal anatomical views of a target volume of tissue to be stimulated, consistent with the present inventive concepts.

FIG. 7 illustrates an anatomical view of a portion of a patient's brain with a distal portion of a stimulation lead implanted to stimulate the fornix, consistent with the present inventive concepts.

FIG. 8 illustrates a side sectional image of a patient's brain with an implanted series of electrodes, consistent with the present inventive concepts.

### DETAILED DESCRIPTION OF THE DRAWINGS

The terminology used herein is for the purpose of describing particular embodiments and is not intended to be limiting of the inventive concepts. As used herein, the singular forms “a,” “an” and “the” are intended to include the plural forms as well, unless the context clearly indicates otherwise.

It will be further understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be understood that, although the terms first, second, third etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element or intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g., “between” versus “directly between,” “adjacent” versus “directly adjacent,” etc.).

Spatially relative terms, such as “beneath,” “below,” “lower,” “above,” “upper” and the like may be used to describe an element and/or feature's relationship to another element(s) and/or feature(s) as, for example, illustrated in the figures. It will be understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g., rotated 90 degrees or at other orientations) and the spatially relative descriptors used herein interpreted accordingly.

The term “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

As used herein, the term “wired pathway” shall refer to an energy and/or information transmission pathway including a physical conduit such as a flexible conduit comprising: one or more wires; one or more optical (e.g. light transmitting) fibers; one or more fluid delivery tubes; and combinations of these.

As used herein, the term “wireless” or “wireless pathway” shall refer to an energy and/or information transmission pathway that does not include or otherwise rely on a physical conduit for transmission, such as an electromagnetic or light transmission of energy and/or information that passes through the tissue of a patient without the use of a physical conduit.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

For example, it will be appreciated that all features set out in any of the claims (whether independent or dependent) can be combined in any given way.

The systems, devices and methods of the present inventive concepts are applicable to treat a patient, such as to treat one or more cognitive diseases and/or disorders of a patient. The cognitive diseases and/or disorders include but are not limited to: Alzheimer's Disease (AD) such as Mild or Moderate Alzheimer's Disease; probable Alzheimer's Disease; a genetic form of Alzheimer's Disease; Mild Cognitive Impairment (MCI); hippocampal damage such as hippocampal damage due to Alzheimer's disease, anoxia, epilepsy or depression; neuronal loss; neuronal damage; chemotherapy induced memory impairment; epilepsy; a seizure disorder; dementia; amnesia; a memory disorder such a spatial memory disorder; traumatic brain injury; cognitive impairment associated with Schizophrenia; Parkinson's Disease related cognitive impairment or dementia; and combinations of these. Additionally or alternatively, the patient can be selected to treat negative symptoms of a disease or disorder selected from the group consisting of: schizophrenia; depression; other conditions of reversible impaired memory or cognition; and combinations of these.

In some embodiments, the patient is selected for treatment as described in applicant's co-pending U.S. application Ser. No. 13/655,652, entitled “Deep Brain Stimulation of Memory Circuits in Alzheimer's Disease”, filed Oct. 19, 2012, the contents of which is incorporated herein by reference in its entirety.

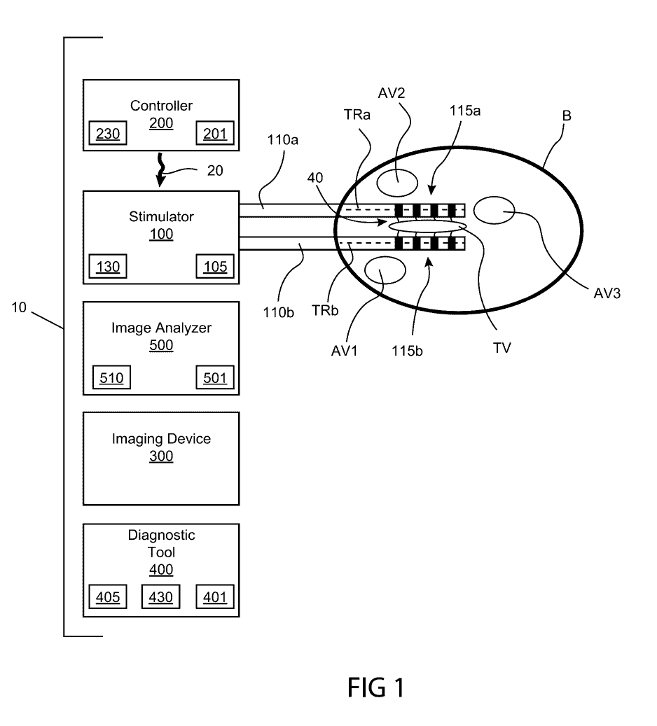

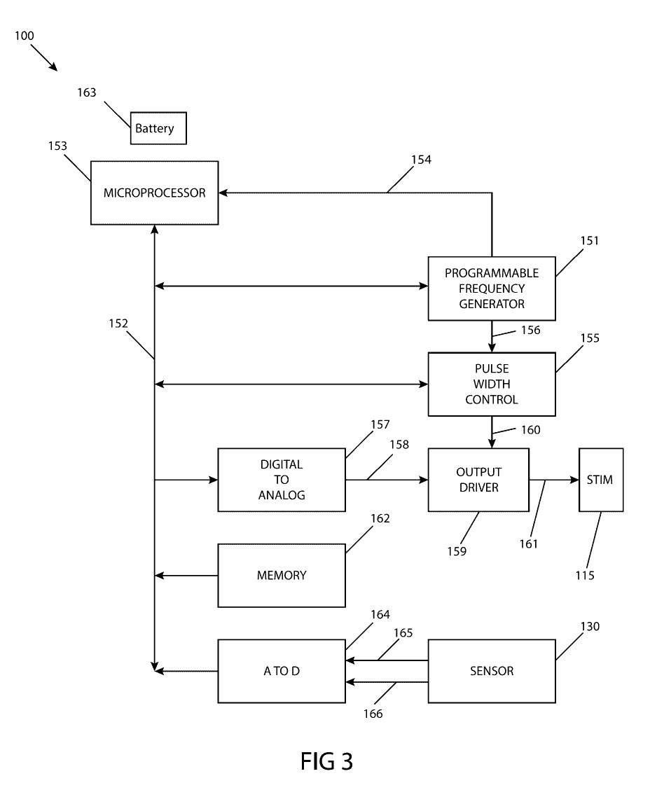

Referring now to FIG. 1, a system for stimulating a patient's brain is illustrated, consistent with the present inventive concepts. System 10 comprises a stimulator 100 and an image analysis device, image analyzer 500. System 10 can be configured to treat a neurological disease, a neurological disorder and/or another patient disease or disorder, as described herebelow. In some embodiments, system 10 is configured to treat multiple neurological diseases, multiple neurological disorders and/or at least one neurological disease and at least one neurological disorder. Stimulator 100 can be configured to stimulate tissue, such as to stimulate one or more portions of a patient's brain B, such as one or more target volumes TV (one shown). In some embodiments, stimulator 100 is configured as described in reference to stimulator 100 of FIG. 3 described herebelow. Stimulator 100 comprises one or more stimulation leads 110 which can be placed into brain B at a trajectory, such as trajectories TRa and TRb shown (singly or collectively trajectory TR). Image analyzer 500 is configured to receive a patient image and identify one or more proposed trajectories TR for implantation of one or more stimulation leads 110, based on one or more target volumes TV of brain tissue to be stimulated. Alternatively or additionally, image analyzer 500 is configured to identify the one or more proposed stimulation lead trajectories TR based on one or more volumes of tissue that the one or more proposed trajectories TR should avoid (avoidance volumes AV described herebelow). In some embodiments, image analyzer 500 is configured to identify the one or more proposed stimulation lead trajectories TR based on both the one or more target volumes TV and the one or more avoidance volumes AV. The one or more stimulation lead trajectories TR can each comprise a path with a starting point (e.g. a point on the skull or a point on the surface of brain B), and an end point (e.g. a point within brain B beyond which the stimulation lead should not advance).

In some embodiments, system 10 comprises controller 200 as shown, a component of system 10 configured to modify the stimulation energy or another stimulation parameter 105 of stimulator 100. In some embodiments, a stimulation parameter 105 comprises a stimulation parameter selected from the group consisting of: an electromagnetic energy stimulation parameter; a light stimulation parameter; a sound stimulation parameter; an agent delivery stimulation parameter; and combinations of these.

In some embodiments, system 10 comprises an imaging component configured to produce patient image information, such as imaging device 300 shown. Imaging device 300 can be configured to provide information related to one or more target volumes TV and/or one or more avoidance volumes AV. In some embodiments, system 10 comprises a diagnostic tool such as diagnostic tool 400 shown. In some embodiments, system 10 is configured such that communication (e.g. wired or wireless communication) can occur between two or more of: stimulator 100, controller 200, diagnostic tool 400 and/or image analyzer 500.

Stimulator 100 can comprise one or more implanted components (e.g. one or more discrete or otherwise physically separated components), one or more components external to the patient P's body, or both at least one implanted component and at least one external component. Stimulator 100 can comprise two or more components, such as two or more components connected with a physical cable including electrically conductive wires and/or optical fibers, or two or more components which transmit and/or receive information via wireless transmission. In some embodiments, stimulator 100 and/or implanted stimulation leads 110 are configured as is described in applicant's co-pending U.S. patent application Ser. No. 13/655,652, entitled “Deep Brain Stimulation of Memory Circuits in Alzheimer's Disease”, filed Oct. 19, 2012, the contents of which is incorporated herein by reference in its entirety.

Stimulator 100 can comprise one or more stimulation leads, such as leads 110a and 110b (singly or collectively leads 110). Each lead 110 comprises one or more stimulation elements, such as stimulation elements 115a of lead 110a and stimulation elements 115b of lead 110b (singly or collectively stimulation elements 115). Stimulation elements 115 can be positioned proximate the distal end of a stimulation lead 110, such as on a distal portion of stimulation lead 110.

Stimulator 100 and one or more stimulation leads 110 can be configured to deliver energy to tissue, via pathway 40 shown. Pathway 40 can comprise a wired or wireless pathway. Stimulation energy delivered can comprise energy selected from the group consisting of: electrical energy; magnetic field energy; light energy; optogenetic energy; sound energy; chemical energy such as energy or other therapeutic benefit provided by a pharmaceutical agent; and combinations of these. Alternatively or additionally, one or more stimulation elements 115 can be configured to deliver a pharmaceutical, chemical or other agent (e.g. with or without simultaneous or sequential energy delivery). In these embodiments, stimulator 100 can comprise a drug or other agent delivery pump and one or more stimulation leads 110 can comprise a fluid delivery channel providing the agent to be delivered by one or more stimulation elements 115 (e.g. when stimulation element 115 comprises an opening through which the agent is delivered). One or more stimulation elements 115 can comprise a stimulation element selected from the group consisting of: an electrode such as one or more electrodes configured to deliver electrical stimulation energy; a magnetic field delivery element; a light delivery element such as a visible, ultraviolet or infrared light delivery element; an optogenetic delivery element; a sound delivery element such as a subsonic wave or ultrasound wave delivery element; an agent delivery element such as a chemical or pharmaceutical agent delivery element; and combinations thereof.

In some embodiments, stimulator 100 and one or more stimulation leads 110 are configured to deliver electrical energy to one or more target volumes TV (e.g. via pathway 40). In these embodiments, one or more stimulation leads 110 can comprise one or more stimulation elements 115 that include an electrode. Stimulations elements 115 can comprise an array of two or more electrodes in a linear arrangement, such as a linear array of four stimulation elements 115 as shown in FIG. 1. Electrical energy delivered by one or more stimulation elements 115 can comprise one or more electrical signals provided at a relatively constant voltage or a constant current, each in a continuous and/or pulsed pattern. In some embodiments, electrical energy is provided by one or more stimulation elements 115 in a pulsed pattern of voltage with a magnitude between 2.0 volts and 10.0 volts, such as a magnitude of approximately 3.0 volts to 3.5 volts. In some embodiments, electrical energy is provided by one or more stimulation elements 115 in a pulsed manner, such as in a continuous series of approximately 90μ seconds at approximately 130 Hz. In some embodiments, one or more stimulation elements 115 deliver electrical energy as described herebelow in reference to FIG. 3.

As described above, stimulator 100 can comprise an implanted portion and/or a portion external to the patient's skin. One or more stimulation leads 110 can comprise a lead placed through the skin (e.g. to attach at its proximal end to an external portion of stimulator 100), or can be fully implanted. When fully implanted, one or more leads 110 can to attach to an implanted portion of stimulator 100, such as when stimulator 100 is implanted in the chest of the patient and leads 110 pass under the skin up to the skull, through the skull (e.g. through a burr hole) and into brain B. Stimulation elements 115 are placed into brain B tissue proximate a target volume TV of tissue to be stimulated. In some embodiments, target volume TV can comprise a single continuous volume of tissue, such as a portion of the fornix of brain B. In other embodiments, target volume TV can comprise multiple discrete (e.g. discontinuous) volumes of tissue such as a portion of the fornix and a portion of the entorhinal cortex. In some embodiments, target volume TV comprises tissue selected from the group consisting of: fornix; entorhinal cortex; hippocampus; anterior thalamic nucleus; amygdala; mammilary bodies; parahippocampal cortex; temporal neocortex; septal nuclei; nucleus basalis of Meynert; subcallosal or subgenual cingulate; ventral capsule; ventral striatum and combinations thereof. In some embodiments, target volume TV comprises tissue selected from the group consisting of: Papez Circuit; hippocampus; cingulate gyrus; fornix; a mammilothalamic tract; amygdala; hypothalamus; mammilary bodies; septal nuclei; temporal neocortex; the medial forebrain bundle; anterior and mediodorsal nuclei of the thalamus; the diagonal band of the Broca; temporal stem and temporal white matter; brainstem; nucleus basalis of Meynert; anterior thalamic nucleus; entorhinal cortex; rhinal cortex; periventricular zone; anterior thalamus; anterior insula; caudate; dorsal anterior cortex; dorsal cingulate; medial frontal cortex; nucleus accumbens; orbital frontal cortex; parietal region; periaqueductal gray area; posterior cingulate area; subcallosal area; subcallosal cingulate; subgenual cingulate; Brodmann area 10; Brodmann area 24; Brodmann area 25; Brodmann area 11/Brodmann area 10; Brodmann area 24b; Brodmann area 31; Brodmann area 32/Brodmann area 10; Brodmann area 32/Brodmann area 11; Brodmann area 39; Brodmann area 46; Brodmann area 46/Brodmann area 9; Brodmann area 47; Brodmann area 6; Brodmann area 9; ventral/medial prefrontal cortex area; ventral/medial white matter; dorsolateral prefrontal cortex; premotor cortex; ventrolateral prefrontal cortex; dorsal anterior cingulate caudate nucleus; frontal pole periaqueductal gray area; dorsolateral prefrontal area; subsingular cingulate; parahippocampal cortex; parahippocampal gyrus; ventral capsule; ventral striatum; anterior commissure; corpus callosum; and combinations thereof.

Stimulator 100 can take the form of a fully implanted signal generator, such as a signal generator similar to signal generator Model 7424, manufactured by Medtronic, Inc. under the trademark Itrel II. Stimulation leads 110 can comprise one or more forms, such as any of the leads compatible with the Model 7424 such as Model 3387 lead set, for stimulating brain B. The lead can be coupled to stimulator 100 by a compatible lead extension.

In some embodiments, stimulation lead 110 comprises up to four implanted stimulation elements 115, such as four electrodes implanted into a portion of brain B using conventional stereotactic surgical techniques and/or “frameless” implantation techniques. In some embodiments, stimulation elements 115 comprise two or more electrodes spaced approximately 1.5 mm apart. Stimulation leads 110 can be individually connected to stimulator 100. System 10 can comprise twin leads, such as leads 110a and 110b shown. Both leads 110a and 110b can be connected to a single component stimulator 100. Alternatively, lead 110a can be connected to a first stimulator 100 component and lead 110b can be connected to a separate, second stimulator 100 component (e.g. a first implanted portion in the left side of the patient's chest and a second implanted portion in the right side of the patient's chest). In these dual stimulation lead embodiments, stimulation elements 115a can be placed on one side of a target volume TV and stimulation elements 115b can be placed on another (e.g. opposite) side of a target volume TV, such as to allow bilateral stimulation of the target volume TV (e.g. bilateral stimulation of the fornix or other brain B structure). Alternatively, stimulation elements 115a can be placed proximate a first target volume TV and stimulation elements 115b can be placed proximate a separate, second target volume TV, such as to stimulate two different target volumes TV.

In some embodiments, leads 110 comprise two or more electrodes, such as two electrodes positioned in two separate nuclei that potentiate each other's effects. In some embodiments, stimulation elements 115 comprise two electrodes in two separate nuclei with opposite effects, with the stimulation delivered being used to fine-tune the response through opposing forces. It will be appreciated, however, that any number of electrodes or other stimulation elements 115 can be positioned within brain B, on or proximate to brain B, remote from brain B, and/or external to the patient P's body, in accordance with the present inventive concepts. Additionally, one or more secondary electrodes or secondary stimulation elements can be implanted or otherwise positioned so that a secondary stimulation portion lies in communication with another predetermined portion of brain B.

System 10 can be utilized in monopolar and/or multipolar electrical stimulation configurations (e.g. monopolar, bipolar and/or stimulation configurations including three or more poles). In some embodiments, system 10 delivers monopolar energy, such as when a housing of at least a portion of stimulator 100 is implanted in the patient, such that the housing can function as a lead (e.g. a positive lead). In these embodiments, stimulation element 115 can comprise one or more electrodes positioned in brain B proximate a target volume TV, the one or more electrodes functioning as the associated lead (e.g. as negative leads).

System 10 can be configured to provide stimulation continuously and/or intermittently, such as for a chronic period of time of at least 1 month, at least 3 months or at least 6 months. In some cases, stimulation can be provided for a longer period of time such as 12 months or more. Intermittent stimulation can include delivery of constant or pulsed stimulation energy with stimulation “on” times of at least 30 minutes, or at least 60 minutes. In some embodiments, the constant or pulsed stimulation energy delivery duty cycle (ratio of “on” time to the sum of “on” time plus “off” time) ranges from 20% to 80%. Stimulation can be performed in either an open loop or closed loop mode. In some embodiments, stimulation is initiated and/or modified to achieve an acute goal (e.g. by a caregiver or the patient), such as to perform an acute task or activity in which enhanced memory function is desirable. Stimulation can comprise delivery of electrical energy, sound energy, chemical energy, light energy, and/or the delivery of a pharmaceutical drug or other agent. Stimulation elements 115 when configured as electrodes can be of various forms selected from the group consisting of: single component bipolar electrode; multiple unipolar electrodes; stacked contact electrodes; discrete electrodes; electrode strip; grid of electrodes; paddle electrode; high-density/high channel or lead count micro-electrodes; and combinations of these.

Stimulator 100 can include an agent delivery mechanism, such as a mechanism including a pump and one or more catheters configured to deliver one or more agents to one or more brain B (e.g. target volume TV) or other body locations. In some embodiments, system 10 is configured to deliver both electrical stimulation and agent delivery, sequentially and/or simultaneously. In these embodiments, a pump can be implanted below the skin of the patient, such as when the pump has an access port into which a needle can be inserted through the skin to inject a quantity of a liquid agent, such as a medication or other drug. The liquid agent is delivered from the pump through a catheter (e.g. after traveling from a pumping chamber and through a catheter access port attached to the side of the pump), and into the patient. The catheter can be positioned to deliver the agent to one or more specific infusion sites of brain B (e.g. one or more target locations TV). The pump can take the form of any number of known implantable pumps including for example that which is disclosed in U.S. Pat. No. 4,692,147, “Drug Administration Device”, the contents of which is incorporated herein by reference in its entirety. Lead 110 can comprise a catheter or other hollow tube, and stimulation element 115 can comprise a distal portion (e.g. the distal end) of the catheter. Lead 110 can be implanted, such as by conventional stereotactic or frameless surgical techniques, into a portion of brain B to affect one or more target volumes TV. Lead 110 can be surgically implanted through a hole in the skull and be positioned between the skull and the scalp, with lead 110 being fluidly attached to a pumping portion of stimulator 100. Stimulator 100 can be implanted in a subcutaneous pocket located in the chest below the clavicle. Alternatively, stimulator 100 can be implanted in the abdomen. Stimulation lead 110 can comprise twin tubes (e.g. two separate catheters attached to a single pump or a single catheter with two lumens) that have their distal portions implanted into brain B in bilateral locations. Alternatively, a second catheter can be implanted on the other side of brain B and can be supplied with drugs or other stimulating agents from a separate pump. Stimulator 100 can be programmed (e.g. via controller 200) to deliver one or more agents according to a particular dosage and/or time interval. For example, stimulator 100 can deliver drug therapy over a first period with a high dose configured to induce a high level of neurogenesis, after which a lower dose is delivered to maintain neurogenesis and secondary trophic effects (e.g. axonal sprouting and synaptogenesis). Any number of neurotrophins or drugs that stimulate neurons can be administered including, but not limited to: NGF; BDNF; NT-3; FGF; EGF; GDNF; Neurteurin; Artemin; Persephin; and combinations of these.

System 10 can be configured to modulate memory circuits to produce clinical benefits, such as to modulate memory circuits in the brain B of patient P to reduce the progression of or otherwise treat the effects of Alzheimer's Disease (AD). System 10 can modulate memory circuits in brain B via electrical or other stimulation means. System 10 can be configured to stimulate brain B tissue selected from the group consisting of: fornix; entorhinal cortex; hippocampus; anterior thalamic nucleus; amygdala; mammilary bodies; parahippocampal cortex; temporal neocortex; septal nuclei; nucleus basalis of Meynert; subcallosal or subgenual cingulate; ventral capsule; ventral striatum; other brain B tissue locations as described herein; and combinations thereof. The stimulation site within one or more locations of brain B tissue can be used to stimulate, activate or otherwise affect one or more similar or different brain B tissue locations, such as a stimulation configured to affect a brain B location selected from the group consisting of: fornix; hippocampus; parahippocampal gyrus; entorhinal cortex; amygdale; mammilary bodies; parahippocampal cortex; temporal neocortex; septal nuclei; nucleus basalis of Meynert; subcallosal or subgenual cingulate; other brain B tissue locations as described herein; and combinations of these. Alternatively or additionally, system 10 and one or more stimulation elements 115 can be constructed and arranged to stimulate non-brain tissue, such as nerve or organ tissue separate from brain B. Stimulated tissue can comprise tissue selected from the group consisting of: vagus nerve; trigeminal nerve; carotid sinus; spinal cord; dorsal root ganglia; tibial nerve; sacral nerve; gastric nerve; and combinations thereof. In some embodiments, system 10 is configured to stimulate at least a portion of the hypothalamus, such as at least a portion of the fornix. The fornix is a large axonal bundle that constitutes a major inflow and output pathway from the hippocampus and medial temporal lobe. The hippocampus is a critical component of the limbic circuitry and is distinguished among some of the regions of the brain by persistent production of new neurons. The fornix is involved in memory formation and is known to be affected early in the progression of AD. In some embodiments, loss of fornix integrity associated with hippocampal volume loss can be detected by diagnostic tool 400 and used by system 10 to predict the progression of AD.

System 10 can be configured to sustain and/or improve the function of the fornix. Alternatively or additionally, system 10 can be configured to therapeutically affect the hippocampus and/or cortical circuits (e.g. the cortico-cortico circuits). Stimulation of the fornix by system 10 can be used to activate the hippocampus and cortical regions in brain B's default network, a network of brain B regions that are active when the individual is not focused on the outside world and/or brain B is at wakeful rest. Patients with AD can exhibit a decrease in glucose metabolism over time. System 10 can be configured to increase or maintain (e.g. prevent the decrease of) glucose metabolism, such as by stimulating at least the fornix. System 10 can be configured to increase or maintain (e.g. prevent the decrease of) one or more portions of hippocampal volume, such as by stimulating the fornix. In some embodiments, the stimulation of system 10 results in neurogenesis, such as hippocampal neurogenesis.

System 10 can be configured to produce clinical benefits to patient P by modulating neurophysiologic activity in pathological circuits. The pathological circuits can be causing functional impairment in the neural elements and circuits underlying cognitive and/or memory functions, and the stimulation provided by system 10 can improve clinical and/or neurobiological outcomes that result from these pathological circuits. Stimulation provided by system 10 can be used to modulate dysfunctional networks, such as to therapeutically manipulate the levels of one or more deleterious proteins.

System 10 can be configured to drive activity in projection structures downstream from the stimulation site (e.g. downstream from the fornix). System 10 can be configured to provide evoked responses that are unequivocal and/or consistent. Stimulation received by system 10 can activate the cingulated gyrus and precuneus area of the parietal lobe, including direct and trans-synaptic sequential activation of downstream targets related to the connectivity of the fornix and hippocampus.

System 10 can be configured to regulate the level of one or more neurotrophic factors and/or neurotransmitters. System 10 can be configured to ameliorate cognitive decline associated with dementia. A patient receiving therapy from system 10 can have reduced integrity of white matter tracts innervating limbic structures such as the fornix (e.g. at least the fornix) as determined by fractional anisotropy maps using diffusion tensor imaging. System 10 can be configured to achieve at least one of: treats memory impairment; improves memory function; treats cognitive function loss; reverses synaptic loss; improves cognitive function; reduces degradation of cognitive function; promotes neurogenesis in the hippocampus of patient P's brain B; drives neurotrophin expression; regulates one or more biomarkers related to Alzheimer's Disease such as amyloid-beta, tau, and/or phosphorylated tau; regulates BDNF expression; increases neurotransmitter release such as acetylcholine; or improves glucose utilization in the temporal lobe, the parietal lobe or both lobes of the patient's brain B.

In some embodiments, a combination of treatment therapies can be delivered to provide influencing of multiple neuronal types. Stimulator 100 can be configured to deliver multiple therapies, such as two or more stimulation therapies selected from the group consisting of: electrical stimulation; magnetic stimulation; optical stimulation (e.g. visible, ultraviolet and/or infrared light); sound stimulation (e.g. ultrasound or subsonic waves); chemical stimulation (e.g. a drug or other agent); and combinations of these, such as described hereabove. For example, it can be desirable to concurrently influence, via chemical, electrical and/or other stimulation, the neurons in the fornix, hippocampus and/or other portions of brain B to achieve an improved result. Such a system 10 utilizing multiple forms of treatment therapy can be similar to that which is disclosed, for example, in U.S. Pat. No. 5,782,798. In addition to affecting the deep brain, it can be desirable for system 10 to affect concurrently other portions of brain B.

In some embodiments, system 10 is configured to provide one or more pharmaceutical or other agents, such as an agent delivered orally, via an injection, or delivered by a component of system 10. In some embodiments, system 10 is configured to provide a cholinesterase inhibitor medication or other agent to patient P. Stimulation element 115a and/or 115b can be constructed and arranged to deliver one or more pharmaceutical or other agents, such as when stimulation element 115a and/or 115b are further configured as a drug delivery element or other liquid or solid dispensing element.

Controller 200 can be configured to initiate and/or adjust (hereinafter “set” or “setting”) one or more stimulation parameters 105 of stimulator 100, such as one or more test stimulation parameters and/or one or more treatment stimulation parameters (collectively or singly referred to as “stimulation parameters”). Controller 200 can be configured to communicate with stimulator 100, via pathway 20, such as to set one or more stimulation parameters 105 of stimulator 100. Pathway 20 can comprise a wired or wireless pathway as described herein.

Controller 200 can be configured to initiate, adjust and/or otherwise set at least one stimulation parameter 105, such as a stimulation parameter selected from the group consisting of: voltage level such as an average voltage level, rms voltage level and/or a peak voltage level; current level such as an average current level, rms current level and/or a peak current level; power level such as an average power level, rms power level and/or a peak power level; frequency of stimulation signal; series of frequencies of the stimulation signal; phase of stimulation signal; pulse width modulation ratio; signal pulse width; current density such as current density applied to tissue; single electrode selected to receive stimulation energy; set of electrodes selected to receive monopolar and/or bipolar stimulation energy; agent delivery rate; physiologic concentration of an agent to be delivered; power of light delivered to tissue; frequency of light delivered to tissue; a modulation parameter of light delivered to tissue; amplitude of sound delivered to tissue; frequency of sound delivered to tissue; a modulation parameter of sound delivered to tissue; mass of agent delivered to tissue; volume of agent delivered to tissue; concentration of agent delivered to tissue; delivery rate of agent delivered to tissue; and combinations of these. System 10 stimulation parameters 105 can be set by signals sent from controller 200 to stimulator 100 via pathway 20.

Controller 200 comprises user interface 201, such as a user interface configured to provide information to and/or receive commands from an operator of system 10. User interface 201 can comprise one or more user input and/or user output components selected from the group consisting of: a touchscreen; a graphical and/or alphanumeric screen; a keypad; a mouse; and combinations of these. Controller 200 can comprise one or more discrete controllers, such as one or more handheld devices configured to program or otherwise communicate with stimulator 100, imaging device 300, diagnostic tool 400 and/or image analyzer 500. Pathway 20 can comprise a uni-directional or bi-directional communication pathway between controller 200 and stimulator 100. Pathway 20 can comprise one or more physical conduits such as electrically conductive wires and/or optical fibers. Alternatively or additionally, pathway 20 can comprise a wireless communication pathway, such as a transmission of electromagnetic waves such as is used in wireless radiofrequency (RF) communications.

Diagnostic tool 400 can be configured to measure one or more patient parameters, and to produce diagnostic data 405 representing the measured patient parameters. The measuring of diagnostic data 405 by diagnostic tool 400 can include but is not limited to performing a data measurement function selected from the group consisting of: recording; gathering; assessing; collecting; determining; processing; combining; and combinations of these. System 10 can be configured such that stimulator 100 delivers test stimulation energy to brain B based on one or more test stimulation parameters. System 10 can be further configured to deliver treatment stimulation energy to brain B based on one or more treatment stimulation parameters, such as when the treatment stimulation parameters are based on the diagnostic data 405 produced by diagnostic tool 400. In some embodiments, system 10, stimulator 100 and/or diagnostic tool 400 are configured as described in reference to applicant's co-pending International PCT Application Serial Number PCT/US2014/060923, entitled “Brain Stimulation Systems including Diagnostic Tool”, filed Oct. 16, 2014, the content of which is incorporated herein be reference in its entirety.

Diagnostic tool 400 can be configured to record, gather, assess, collect, determine and/or otherwise measure one or more patient parameters and produce diagnostic data 405 representing these one or more patient parameters. Diagnostic tool 400 can be further configured to process (e.g. mathematically process) and/or combine measured data, such as when diagnostic tool 400 comprises one or more algorithms configured to analyze diagnostic data 405, such as one or more algorithms that compare diagnostic data 405 to one or more “stimulation thresholds” and record one or more stimulation parameters 105 associated with the one or more stimulation thresholds. In some embodiments, an algorithm is configured to determine a stimulation threshold correlating to an undesired clinical event or other undesired patient event (hereinafter “adverse event”) as described herein. In some embodiments, an algorithm is configured to determine a stimulation threshold correlating to a desired clinical event or other desired patent event (hereinafter “desired event”), such as an event in which a desired memory recall occurs, a desired memory learning is achieved and/or other desired event occurs. In some embodiments, a stimulation parameter 105 is set at a level at or above (hereinafter “above”) a stimulation threshold that caused a desired event (e.g. as determined in a diagnostic test of the present inventive concepts). In these embodiments, the term “above” does not necessarily correlate to a higher magnitude of stimulation energy, but represents a higher, lower or similar value that tends toward causing occurrence of the desired event.

Diagnostic tool 400 can comprise a user interface 401, such as a user interface configured to provide information to and receive commands from an operator of system 10. User interface 401 can comprise one or more user input and/or user output components selected from the group consisting of: a touchscreen; a graphical and/or alphanumeric screen; a keypad; a mouse; and combinations thereof. As described above, diagnostic tool 400 can be configured to measure one or more patient parameters and produce diagnostic data 405 which is determined based on the one or more measured patient parameters. Diagnostic data 405 can be displayed on user interface 401 (such as heart rate information, blood pressure information, or other data corresponding to a measured patient parameter that is displayed on user interface 401). In some embodiments, diagnostic tool 400 can communicate directly with controller 200, stimulator 100, such as via a wired or wireless connection as described herein. Diagnostic data 405 can be recorded by controller 200 and/or stimulator 100, such as to automatically and/or semi-automatically modify one or more stimulation parameters 105. Diagnostic data 405 can be recorded by image analyzer 500, such as to determine one or more trajectories TR of the present inventive concepts.

As described herein, diagnostic data 405 can be used to determine if an adverse event has occurred or is about to occur. Stimulation parameters 105 can be set at a level below or otherwise different than the stimulation threshold at which the adverse event occurred, such as at a safety margin below or otherwise away from that stimulation threshold (e.g. a voltage or current level that is less than the level causing the adverse event). In some embodiments, one or more stimulation parameters 105 are modified based on a stimulation threshold (e.g. modified to a level at or below the stimulation threshold, such as at a safety margin below the stimulation threshold at which an adverse event occurred). For example, an adverse event that occurs at a signal voltage of 6 Volts, can result in delivering therapy at 5 Volts (a 16.6% safety margin), at 4 Volts (a 33.3% safety margin) or at 3 Volts (a 50% safety margin).

Diagnostic tool 400 can comprise one or more diagnostic devices, such as one or more devices selected from the group consisting of: heart rate monitor; EKG measurement device; oximeter; combined heart rate and oximeter device such as a pulse oximeter; blood pressure measurement device; neuronal activity measurement device; EEG measurement device; evoked response potential (ERP) measurement device; neurochemical analysis device; memory test device; memory test form; respiration measurement device; sweat measurement device; skin conductivity measurement device; pH measurement device; body motion measurement device; imaging device; and combinations of these. Diagnostic tool 400 can be configured to detect and/or record an adverse event, such as an adverse event selected from the group consisting of: undesirable heart rate; undesirable respiration rate; undesirable sweating; undesirable hallucinations; undesirable tingling; flushing; undesirable psychiatric effect; undesirable cognitive effect; unpleasant generalized warming; undesirable perceptions described as déjá vu; seizure; synchronized neuronal firing pattern; undesired neural response time; undesired brain state; undesired theta phase; undesired p300 amplitude; and combinations of these.

In some embodiments, diagnostic tool 400 comprises two independent diagnostic measurement devices, for example two devices whose diagnostic data are used in combination. For example, diagnostic tool 400 can comprise a blood pressure measurement device and a heart rate measurement device, such as to identify patient discomfort or other patient issue (e.g. a falsehood or other inaccurate statement made by the patient that can be detected through analysis of a patient parameter such as heart rate and/or blood pressure).

Stimulator 100 can comprise one or more sensors, such as sensor 130 shown. Controller 200 can comprise one or more sensors, such as sensor 230 shown. Diagnostic tool 400 can comprise one or more sensors, such as sensor 430 shown. Sensors 130, 230 and/or 430 can comprise a sensor selected from the group consisting of: neuronal activity sensor; EEG sensor; local field potential sensor; neurochemical sensor; pH sensor; pressure sensor; blood pressure sensor; optical sensor; blood gas sensor; blood oxygen sensor; magnetic sensor; strain gauge; temperature sensor; and combinations of these. Sensor 130, 230 and/or 430 can each comprise an implanted or external sensor.

System 10 can be configured to provide open loop stimulation to brain B. Alternatively or additionally, system 10 can be configured to provide closed loop stimulation to brain B, such as closed loop stimulation based on diagnostic data 405 provided by diagnostic tool 400 and/or a signal provided by one or more of sensors 130, 230, 330 and/or a separate implanted or external sensor.

Imaging device 300 can comprise a device selected from the group consisting of: MRI; fMRI; X-ray; fluoroscope; Ct-Scanner; PET Scanner; Diffusion Tensor Imaging device; ultrasound imaging device; and combinations thereof. Imaging device 300 can provide patient images, such as patient brain B images to be displayed and/or analyzed by image analyzer 500. Imaging device 300 can provide multiple views of the patient's brain B, such as sectional views and two views which are orthogonal to each other. In some embodiments, imaging device comprises a series of images or other anatomical data to provide a three dimensional model of at least the patient's brain B. In some embodiments, an MRI or other image is created with the use of a contrast injection. In some embodiments, T1-weighted images are created using an MRI.

Image analyzer 500 comprises a user interface 501 and a display 510. User interface 501 can comprise one or more user input and/or user output components selected from the group consisting of: a touchscreen; a graphical and/or alphanumeric screen; a keypad; a mouse; and combinations thereof. Image analyzer 500 is configured to provide one or more patient images on display 510, such as one or more patient images recorded by imaging device 300. One or more proposed trajectories TR can be shown on display 510.

Image analyzer 500 can be configured to allow an operator to select (e.g. a clinician via user interface 501) one or more target volumes TV, such as the one or more target volumes described herein. Image analyzer 500 can be further configured to allow an operator to identify (e.g. via user interface 501), one or more avoidance volumes of tissue. The avoidance volumes of tissue can comprise tissue through which stimulation lead 110 should not pass and/or volumes of tissue that should receive minimal or no stimulation from a stimulation element 115. In some embodiments, an avoidance volume AV (e.g. AV1, AV2 and/or AV3 shown in FIG. 1) comprises a blood vessel, such as a blood vessel selected from the group consisting of: an artery of brain B; a vein of the brain B; and combinations of these. In some embodiments, an avoidance volume AV comprises a sulcus of the brain B surface, such as when the initial penetration point avoids the sulcus (e.g. the penetration point is proximate to a middle portion of a gyrus of the brain B surface). An avoidance volume AV can comprise tissue proximate a wall of the ventricle, such as a wall location relatively parallel to a potential directory that can result in stimulation lead 110 sciving the wall (e.g. non-orthogonally passing through a small portion of the wall) and/or deflecting off the wall.

An avoidance volume AV can comprise the optical tract, such as to avoid stimulating the optical tract with a stimulation element 115. An avoidance volume AV can comprise a volume of tissue which when stimulated activates an autonomic or vegetative physiological response detrimental to the health of the patient or a sensory response disagreeable to the patient. An avoidance volume AV can comprise a volume of tissue which when stimulated activates a sensory response unpleasant for the patient. In some embodiments, an avoidance volume of tissue comprises one or more tissue locations selected from the group consisting of: posterior hypothalamic area; ventral tegmental area; lateral hypothalamic area; anterior hypothalamic nucleus; paraventricular nucleus; dorsal medial hypothalamic nucleus; ventromedial hypothalamic nucleus; arcuate nucleus; lateral tuberal nucleus; medial preoptic nucleus; supraoptic nucleus; and combinations thereof. In some embodiments, the target volume comprises at least a portion of the fornix and an avoidance volume comprises the fornix, such as when stimulation elements 115 are positioned proximate a portion of the fornix to stimulate the fornix while no portion of the stimulation lead 110 penetrates the fornix (e.g. to prevent damage to the fornix). In some embodiments, an avoidance volume AV comprises the hypothalamus, such as to avoid stimulating the hypothalamus.

As described above, image analyzer 500 can be configured to identify one or more proposed stimulation lead trajectories TR. In some embodiments, at least one proposed trajectory TR is identified to position at least one stimulation element 115 proximate the target volume TV, such as to optimally or otherwise effectively stimulate target volume TV. Alternatively or additionally, at least one trajectory TR can be identified to avoid stimulation lead 110 from penetrating an avoidance volume AV. The at least one trajectory TR can be identified to avoid stimulation lead 110 from penetrating any portion of the target volume TV, such as when the target volume TV comprises at least a portion of the fornix and the avoidance volume AV comprises at least a portion of the fornix. The avoidance volume AV can comprise tissue selected from the group consisting of: tissue proximate a sulcus; blood vessel tissue; tissue proximate a brain B ventricle wall (e.g. to avoid sciving of the wall and/or undesired deflection of stimulation lead 110); optical tract tissue (e.g. to avoid damage and/or stimulation of optical tract tissue); and combinations of these. Image analyzer 500 can be configured to identify a stimulation lead trajectory TR that avoids one or more avoidance volumes AV by a minimum distance (e.g. a threshold distance). In these embodiments, one or more threshold distances can be set by a clinician or other operator of system 10, such as via user interface 501. In some embodiments, a first threshold distance is set for a first avoidance volume AV1 and a second threshold distance is set for a second threshold distance AV2. The first and second threshold distances can be similar or dissimilar, and either can be set by an operator of system 10.

Image analyzer 500 can be configured to identify one or more stimulation lead trajectories TR based on avoiding penetration of a target volume TV. such as when the target volume TV comprises the fornix and one or more stimulation elements 115 are positioned proximate to the fornix, but stimulation elements 115 and the remainder of stimulation lead 110 are positioned to avoid penetrating the fornix (e.g. to properly stimulate the fornix without damaging it). In some embodiments, a stimulation lead trajectory TR is identified to position all portions of stimulation lead 110 away from the target volume TV by a threshold distance, such as a threshold distance set by an operator of the system. In some embodiments, a stimulation lead trajectory TR is identified to position one or more stimulation elements 115 within 2 mm of the anterior border of the fornix.

Image analyzer 500 can be configured to identify one or more stimulation lead trajectories TR that pass through a gyrus on the surface of the brain B (e.g. avoid a sulcus on the surface of brain B). A trajectory TR can be identified that is relatively parallel with a major axis of the target volume, such as a trajectory TR that is relatively parallel with a major axis of the fornix (e.g. a major axis of an anterior pillar of the fornix). A trajectory TR can be identified that comprises a lateral angle between 8° and 10° from the mid-sagittal plane. A trajectory TR can be identified that comprises an anterior angle between 50° and 60° from the axial plane. A trajectory TR can be identified that passes through the lateral ventricle. A trajectory TR can be identified that avoids or reduced non-linear deflection of the stimulation lead 110, such as a trajectory TR that is proximate (e.g. touching) and parallel to a ventricle wall.

Image analyzer 500 can be configured to identify a set of multiple proposed stimulation lead trajectories TR. The multiple trajectories TR can represent one or more continuous volumes of tissue, such as one or more continuous volumes of tissue each with a triangular and/or trapezoidal cross-sectional geometry (e.g. a cone-shaped volume of tissue).

Image analyzer 500 can be configured to identify one or more proposed stimulation lead trajectories TR that position one or more stimulation elements 115 to avoid stimulating certain tissue volumes (e.g. non-target tissue volumes). In some embodiments, the non-target tissues to reduce or avoid stimulating comprise tissue selected from the group consisting of: hippocampal tissue; optical tract tissue; and combinations thereof. Alternatively or additionally, the non-target tissue to reduce or avoid stimulating can comprise tissue selected from the group consisting of: posterior hypothalamic area; ventral tegmental area; lateral hypothalamic area; anterior hypothalamic nucleus; paraventricular nucleus; dorsal medial hypothalamic nucleus; ventromedial hypothalamic nucleus; arcuate nucleus; lateral tuberal nucleus; medial preoptic nucleus; supraoptic nucleus; and combinations thereof.

Image analyzer 500 can be configured to identify one or more stimulation lead trajectories TR that position the distal end of a stimulation lead 110 approximately 1 cm beyond a ventricle of the brain B, such as when the target volume TV comprises the fornix (e.g. an anterior pillar of the fornix). In these embodiments, the distal end of the stimulation lead 110 can be placed above the optic tract of the brain B, such as approximately 1 mm from the optic tract (i.e. the distal end of the associated trajectory TR is positioned approximately 1 mm from the optic tract).

Image analyzer 500 and user interface 501 can be configured to allow an operator (e.g. a clinician) to perform one or more functions such as a function selected from the group consisting of: select a patient image; rotate an image; magnify a patient image; select a target volume TV; magnify the view of a target volume TV; view an avoidance volume AV; magnify an avoidance volume AV; view one or more stimulation lead trajectories TR identified by image analyzer 500; analyze one or more stimulation lead trajectories TR identified by image analyzer 500; select one or more stimulation lead trajectories TR identified by image analyzer 500; measure the distance between one or more stimulation lead trajectories TR and a target volume TV; measure the distance between one or more stimulation lead trajectories TR and an avoidance volume AV; measure the distance between one or more stimulation elements 115 proposed locations and a target volume TV; measure the distance between one or more stimulation elements 115 proposed locations and an avoidance volume AV; average one or more measured distances; sum two or more measured distances; and combinations of these.

As described hereabove, image analyzer 500 can be configured to identify one or more proposed trajectories TR for placement of one or more stimulation leads 110. A clinician or other operator of system 10 can select one or more of the placement trajectories (TR_{PL}) for actual placement of one or more stimulation leads 110 from the one or more proposed trajectories TR identified by image analyzer 500. In some embodiments, image analyzer 500 is configured to provide an analysis of one or more proposed trajectories TR and/or one or more placement trajectories TR_{PL}. In these embodiments, the analysis can provide a qualitative and/or quantitative measure of the one or more proposed trajectories TR and/or one or more placement trajectories TR_{PL}.

Image analyzer 500 can be configured to provide a qualitative measure of the avoidance of one or more trajectories TR and/or TR_{PL} to one or more avoidance volumes AV, such as a qualitative measure of one or more of: proximity of the a stimulation lead 110 to an avoidance volume AV; and average distance between a stimulation lead 110 and an avoidance volume AV. Alternatively or additionally image analyzer 500 can be configured to provide a qualitative measure of a geometric relationship between one or more trajectories TR and/or TR_{PL} and a target volume TV, such as a qualitative measure of one or more of: proximity of a stimulation element 115 to a target volume TV; average proximity of an array of stimulation elements 115 to a target volume TV; and degree of parallelism of an array of stimulation elements 115 (e.g. a linear array) to a target volume TV. In some embodiments, image analyzer 500 is configured to compare or otherwise assess multiple trajectories, such as one or more proposed trajectories TR identified by image analyzer 500 and/or one or more placement trajectories TR_{PL} selected by an operator of system 10.

Image analyzer 500 can be configured to provide a quantitative measure of the avoidance of one or more trajectories TR and/or TR_{PL} to one or more avoidance volumes AV, such as a quantitative measure of one or more of: proximity of the a stimulation lead 110 to an avoidance volume AV; and average distance between a stimulation lead 110 and an avoidance volume AV. Alternatively or additionally image analyzer 500 can be configured to provide a quantitative measure of a geometric relationship between one or more trajectories TR and/or TR_{PL} and a target volume TV, such as a quantitative measure of one or more of: proximity of a stimulation element 115 to a target volume TV; average proximity of an array of stimulation elements 115 to a target volume TV; and degree of parallelism of an array of stimulation elements 115 (e.g. a linear array) to a target volume TV. In some embodiments, image analyzer 500 is configured to compare or otherwise assess multiple trajectories, such as one or more proposed trajectories TR identified by image analyzer 500 and/or one or more placement trajectories TR_{PL} selected by an operator of system 10.

Image analyzer 500 can be configured to provide a distance, angle or other measurement information regarding the geometric relationship between one or more stimulation leads 110 and one or more target volumes TV. Image analyzer 500 can be configured to provide distance, angle or other measurement information regarding the geometric relationship between one or more stimulation elements 115 and one or more target volumes TV. In some embodiments, image analyzer 500 is configured to allow a clinician or other operator to perform a function selected from the group consisting of: select a patient image; rotate an image; magnify a patient image; select a target volume; magnify the view of a target volume; view an avoidance volume; magnify an avoidance volume; view one or more stimulation lead trajectories identified by the image analyzer; analyze one or more stimulation lead trajectories identified by the image analyzer; select one or more stimulation lead trajectories identified by the image analyzer; measure the distance between one or more stimulation lead trajectories and a target volume; measure the distance between one or more stimulation lead trajectories and an avoidance volume; measure the distance between one or more stimulation elements proposed locations and a target volume; measure the distance between one or more stimulation elements proposed locations and an avoidance volume; average one or more measured distances; sum two or more measured distances; and combinations of these.

One or more components of system 10 can include another component of system 10, such as when one or more of at least a portion of stimulator 100, controller 200, imaging device 300, diagnostic tool 400, and/or image analyzer 500 are combined (e.g. within a common housing). For example, at least a portion of stimulator 100 can comprise at least a portion of controller 200, such as when stimulator 100 includes an external portion comprising user interface 201 which is configured to set one or more stimulation parameters 105. In some embodiments, at least a portion of stimulator 100 can comprise at least a portion of diagnostic tool 400, such as when stimulator 100 comprises one or more sensors 130 constructed and arranged to record one or more patient parameters. In some embodiments, one or more sensors 130 are further constructed and arranged to stimulate tissue such as brain B tissue. In some embodiments, at least a portion of controller 200 comprises at least a portion of diagnostic tool 400, such as when controller 200 comprises one or more sensors 230 configured such that controller 200 can function as a heart rate monitor, a blood pressure monitor and/or other diagnostic tool configured to produce diagnostic data 405. In some embodiments, image analyzer 500 comprises controller 200 (e.g. when image analyzer 500 comprises one or more housings which surround an electronic module configured to provide both image analysis and stimulator control functions).

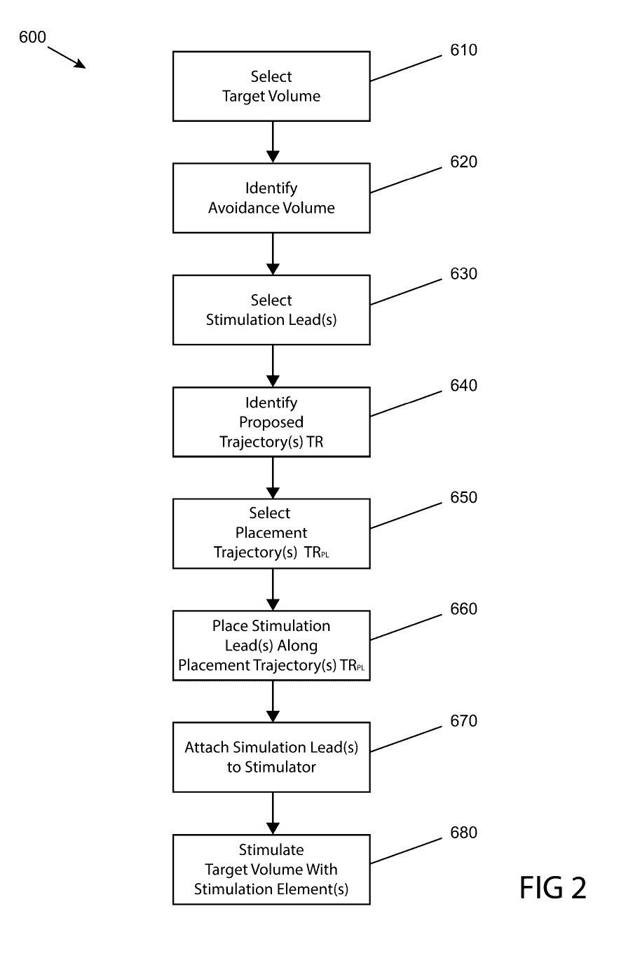

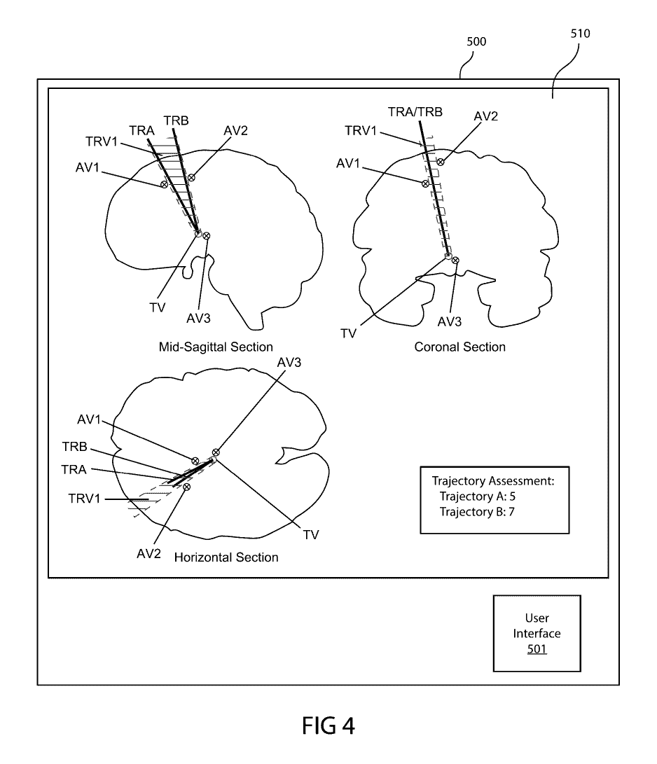

In some embodiments, system 10 is configured to allow a clinician to perform the method of FIG. 2 described herebelow. System 10 can be configured to select one or more simulation lead trajectories TR, such as those described herebelow in reference to FIGS. 4, 5, 6, 7, and 8.

Referring now to FIG. 2, a flow chart of a method for treating a patient with a brain stimulation system is illustrated, consistent with the present inventive concepts. Method 600 comprises STEPs 610 through 680 which include identifying one or more proposed trajectories TR for implantation of one or more stimulation leads, such as leads 110 described hereabove in reference to FIG. 1. STEPs 610 through 680 can be performed using one or more components of system 10 described hereabove in reference to FIG. 1.

One or more patient images (e.g. one or more brain images) can be captured in any of STEPS 610 through 680. The one or more patient images can be obtained using an imaging device, such as an MRI or other imaging device, such as imaging device 300 described hereabove in reference to FIG. 1. In some embodiments, one or more patient images are captured using a contrast injection. In some embodiments, one or more patient images comprise a T1-weighted MRI image.

In STEP 610, one or more target volumes of tissue TV are selected. The selection can be made using one or more patient brain images, such as one or more images provided by imaging device 300. The one or more target volumes of tissue TV can include one or more brain tissue locations, such as those described in detail herein.

In STEP 620, one or more avoidance volumes AV of tissue are selected. The selection can be made using one or more patient brain images, such as one or more images provided by imaging device 300. The avoidance volumes can be selected manually, such as via an operator such as a clinician. Alternatively or additionally, the identifying of one or more avoidance volumes can be performed automatically and/or semi-automatically by image analyzer 500.

In STEP 630, one or more stimulation leads are selected, such as a stimulation lead constructed and arranged similar to stimulation lead 110 described hereabove in reference to FIG. 1.

In STEP 640, one or more proposed trajectories TR for the placement of the stimulation lead are identified. The one or more proposed trajectories TR are identified based on: one or more target volumes TV (e.g. one or more target volume TV locations); one or more avoidance volumes AV (e.g. one or more avoidance volume AV locations); and combinations of these. In some embodiments, the one or more proposed trajectories TR are identified based on both one or more target volumes TV and one or more avoidance volumes AV. In some embodiments, the one or more proposed stimulation lead trajectories TR are identified as described hereabove in reference to FIG. 1.

In STEP 650, one of the proposed trajectories TR is selected for placement of a stimulation lead, from the one or more trajectories TR identified in STEP 640. The placement trajectory TR_{PL} can be selected based on information provided by an image analyzer of the present inventive concepts. The placement trajectory TR_{PL} can be selected based on a qualitative and/or quantitative assessment provided by the image analyzer. The placement trajectory TR_{PL} can be selected based on an assessment of the resultant stimulation of one or more target volumes and/or an assessment of the geometric relationship between a stimulation lead and one or more avoidance volumes. In some embodiments, the placement trajectory TR_{PL} is selected to reduce the distance between one or more stimulation elements and the target volume. In some embodiments, the placement trajectory TR_{PL} is selected to reduce the distance between two or more stimulation elements and the target volume. In some embodiments, the stimulation lead comprises multiple stimulation elements positioned along a first axis, the target volume TV comprises a second axis (e.g. a major axis of the target volume TV), and the placement trajectory TR_{PL} is selected based on increasing the parallelism between the first axis and the second axis. In some embodiments, the placement trajectory TR_{PL} is selected to avoid a stimulation lead from passing through a target volume such as the fornix. In some embodiments, the placement trajectory TR_{PL} is selected to pass through a gyrus of the brain surface. In some embodiments, the placement trajectory TR_{PL} is selected to pass through a lateral ventricle of the brain. In some embodiments, the placement trajectory TR_{PL} is selected to minimize non-linear deflection of a stimulation lead (e.g. avoid being proximate and parallel to a wall of a ventricle).

In STEP 660, one or more stimulation leads are placed (e.g. placed within the patient's brain), such as by using standard lead advancement techniques and equipment, placed through a craniotomy or burr hole well known to those of skill in the art. The one or more stimulation leads can be each placed along a placement trajectory TR_{PL}, such that the distal end of the stimulation lead is coincident with the distal end of the placement trajectory TR_{PL}. In some embodiments, the distal end of a placement trajectory TR_{PL} is positioned at a location selected from the group consisting of: approximately 1 cm beyond a ventricle of the brain; away from the optical tract of the brain such as approximately 1 mm from the optic tract; at a location such that non-target tissue (as described hereabove) is not stimulated; and combinations of these.

In STEP 670, the one or more stimulation leads are attached to a stimulator, such as stimulator 100 described hereabove in reference to FIG. 1. Stimulator 100 can comprise a stimulator implanted in the patient, and the stimulation leads can be tunneled under the patient's skin (e.g. under the skin of the neck) to attach to the implanted stimulator (e.g. a stimulator previously placed in the chest or abdomen of the patient).

In STEP 680, stimulation is delivered by the stimulator via the stimulation lead to the target volume TV, such as by the delivery of electrical energy and/or other stimulation means as described in detail herein. In some embodiments, the stimulation lead comprises one or more stimulation elements selected from the group consisting of: an electrode such as one or more electrodes configured to deliver electrical stimulation energy; a magnetic field delivery element; a light delivery element such as a visible, ultraviolet or infrared light delivery element; an optogenetic delivery element; a sound delivery element such as a subsonic wave or ultrasound wave delivery element; an agent delivery element such as a chemical or pharmaceutical agent delivery element; and combinations thereof.

In some embodiments, STEP 630 comprises selecting multiple stimulation leads, such as multiple stimulation leads 110 described hereabove in reference to FIG. 1. In these embodiments, STEP 640 comprises identifying multiple trajectories TR, STEP 650 comprises selected multiple placement trajectories TR_{PL}, STEP 660 comprises placing the multiple stimulation leads along the associated placement trajectories TR_{PL}, STEP 670 comprises attaching the multiple stimulation leads to one or more stimulators (e.g. one or more stimulators 100 of FIG. 1); and STEP 680 comprises stimulating the target volume TV with the one or more stimulation elements of each of the multiple stimulation leads. In some of these multiple stimulation lead embodiments, the stimulation elements can be positioned on opposite sides of a target volume TV, such as opposite sides of a portion of the fornix. In some of these multiple stimulation lead embodiments, a first stimulation lead can be positioned to stimulate one anterior pillar of the fornix, and a second stimulation lead can be positioned to stimulate the other anterior pillar of the fornix.

Referring now to FIG. 3, a schematic of an electrical stimulation device is illustrated, consistent with the present inventive concepts. Stimulation device 100 delivers electrical stimulation energy including a stimulus pulse frequency that is controlled by programming a value to a frequency generator 151 (e.g. a programmable frequency generator) using bus 152. The frequency generator 151 provides an interrupt signal to microprocessor 153 through an interrupt line 154 when each stimulus pulse is to be generated. The programmable frequency generator 151 communicates with a pulse width control module 155 via pathway 156. The frequency generator 151 can be implemented by a commercial device model CDP1878 sold by Harris Corporation. The amplitude for each stimulus pulse is programmed to a digital to analog converter 157 using bus 152. The analog output is conveyed through a conductor 158 to an output driver circuit 159 to control stimulus amplitude.

Microprocessor 153 also programs pulse width control module 155 using bus 152. The pulse width control module 155 provides an enabling pulse of duration equal to the pulse width via a conductor 160. Pulses with the selected characteristics are then delivered from stimulation device 100 through cable 161 to stimulation element 115. Stimulation element 115, typically comprising one or more electrodes as are described hereabove, can be positioned to stimulate the fornix and/or other regions of the brain or other body tissue. At the time that stimulation device 100 is implanted, an operator of system 10 such as a clinician can program certain key parameters into the memory 162 of the implanted stimulation device 100, such as via telemetry from an external controller, such as controller 200 described in reference to FIG. 1 hereabove. These parameters can be updated subsequently as needed, such as to modify one or more test or treatment stimulation parameters based on diagnostic data produced by a diagnostic device (e.g. diagnostic data 405 produced by diagnostic tool 400 of FIG. 1). Battery 163 can provide electrical power to one or more components of stimulation device 100 described herein.

Stimulation element 115 can comprise one or more deep brain stimulation electrodes, such as electrodes model 3387 produced by Medtronic of Minneapolis, Minn. These electrodes can be bilaterally implanted such that the tips of the electrodes are positioned in a region where cells can be recorded during micro-recording mapping. Alternatively, a single electrode can be implanted unilaterally. Energy is typically applied at a frequency of 2 to 1000 Hz, such as at a frequency of approximately 130 Hz. Energy is typically delivered at a pulse amplitude, such as at a pulse amplitude of approximately 500 μA. Energy is typically delivered at a voltage between 0.1 and 10 Volts, such as between 1 Volt and 6 Volts, such as at a voltage of approximately 3 Volts or approximately 3.5 Volts. Energy delivery can be given in a series of on and off times, such as with an on-time of approximately 30 μseconds to 200 μseconds, such as with an on time of approximately 90 μseconds. The duration of energy delivery can range from 30 minutes to 120 minutes, such as a duration of 60 minutes, which can be repeated at regular or irregular intervals.

The embodiments of the present inventive concepts can be configured as open-loop systems. The microcomputer algorithm programmed by the clinician sets the stimulation parameters of the stimulation device 100. This algorithm can change the parameter values over time but does so independent of any changes in symptoms the patient can be experiencing. Alternatively, a closed-loop system discussed below which incorporates a sensor 130 to provide feedback can be used to provide enhanced results. Sensor 130 (e.g. an implanted or external sensor) can be used with a closed loop feedback system in order to automatically determine the level of electrical stimulation necessary to achieve the desired level of improved cognitive function. In a closed-loop embodiment, microprocessor 153 executes an algorithm in order to provide stimulation with closed loop feedback control. Such an algorithm can analyze a sensed signal and deliver stimulation therapy (e.g. delivery or electrical, magnetic, light, sound and/or chemical treatment therapy) based on the sensed signal. Adjustments can be made when the signal falls within or outside predetermined values or windows, for example, predetermined levels of BDNF and other neurotrophins (e.g., NGF, CNTF, FGF, EGF, NT-3) and corticosteroids. Closed loop applications can be driven by diagnostic data, such as diagnostic data 405 produced by diagnostic tool 400 described in reference to FIG. 1 hereabove.

For example, in some embodiments, the patient can engage in a specified cognitive task, wherein the system measures one or more characteristics to determine if the sensed levels are at expected thresholds. If one or more of the sensed characteristics are outside a predetermined threshold, the system can initiate and/or modify the treatment therapy, such as to enhance or otherwise improve cognitive function.

In some embodiments, the system can be continuously providing closed-loop feedback control. In another embodiment, the system can operate in closed-loop feedback control based on a time of day (e.g., during hours that the patient is awake) or based on a cognitive task (e.g., when the patient is working). In yet another embodiment, the system can be switchable between open-loop and closed-loop by operator control, automatically and/or manually (e.g. manually via a handheld controller).

In some embodiments, the stimulation can be applied before, after and/or during the performance of a memory, cognitive or motor task learning task to facilitate the acquisition of learning or consolidation of the task and in so doing, accelerate the rate of memory acquisition and learning and enhance its magnitude. For example, the stimulation can be provided before, during and/or after periods when the patient is learning a new language or playing a new instrument. Such applied therapy can be useful during the encoding, consolidation and/or retrieval phases of memory. The neuromodulation intervention, brain stimulation via electrical, magnetic, light, sound and/or drug or other agent delivery can occur before, after and/or simultaneous with the memory, cognitive of motor skill task.

In another embodiment, therapy can be provided in relation to learning a task. For example, the stimulation or drug delivery can be applied before, after and/or during the performance of a memory, cognitive or motor task to facilitate the acquisition of learning or consolidation of the task. In so doing, the rate of memory acquisition and learning can be accelerated and enhanced in magnitude. For example, the stimulation or drug delivery can be provided before, during, or after periods when the patient is learning a new language or playing a new instrument. Such therapy can be useful during the encoding, consolidation and/or retrieval phases of memory. The neuromodulation intervention, brain stimulation or drug delivery can occur before, after or simultaneously to the memory, cognitive of motor skill task.

In another aspect of the invention, treatment therapy can be utilized to enhance neurogenesis as a method of improving cognitive function. Techniques for enhancing neurogenesis through treatment therapy are disclosed in co-pending patent application U.S. Ser. No. 14/034,336, entitled “Cognitive Function Within A Human Brain”, filed Sep. 23, 2013; U.S. Ser. No. 14/508,110, entitled “Regulation of Neurotrophins”, filed Oct. 7, 2014; U.S. Ser. No. 14/586,849, entitled “Method Of Treating Cognitive Disorders Using Neuromodulation”, filed Dec. 30, 2014; and in issued U.S. Pat. No. 8,612,006, “Inducing Neurogenesis Within A Human Brain”, issued Dec. 17, 2013; the contents of which are each incorporated herein by reference in their entirety.

Referring back to FIG. 3, the system can optionally utilize closed-loop feedback control having an analog to digital converter 164 coupled to sensor 130 via pathways 165 and 166. Output of the A-to-D converter 164 is connected to microprocessor 153 through peripheral bus 152 including address, data and control lines. Microprocessor 153 processes sensor 130 data in different ways depending on the type of transducer in use and regulates delivery, via a control algorithm, of stimulation based on the sensed signal. For example, when the signal on sensor 130 exceeds a level programmed by the clinician and stored in a memory 162, increasing amounts of stimulation can be applied through an output driver circuit 159. In the case of electrical stimulation, a parameter of the stimulation can be adjusted such as amplitude, pulse width and/or frequency.

Parameters which can be sensed include the activity of single neurons as detected with microelectrode recording techniques, local field potentials, and event related potentials, for example in response to a memory task or sensory stimulus and electroencephalogram or electrocorticogram. For example, U.S. Pat. No. 6,227,203 provides examples of various types of sensors that can be used to detect a symptom or a condition of a cognitive disorder and responsively generate a neurological signal. In an embodiment, a neurochemical characteristic of the cognitive function can be sensed, additionally or alternatively. For example, sensing of local levels of neurotransmitters (glutamate, GABA, Aspartate), local pH or ion concentration, lactate levels, local cerebral blood flow, glucose utilization or oxygen extraction can also be used as the input component of a closed loop system. These measures can be taken at rest or in response to a specific memory or cognitive task or in response to a specific sensory or motor stimulus. In another embodiment, an electro-physiological characteristic of the cognitive function can be sensed. The information contained within the neuronal firing spike train, including spike amplitude, frequency of action potentials, signal to noise ratio, the spatial and temporal features and the pattern of neuronal firing, oscillation behavior and inter-neuronal correlated activity can be used to deliver therapies on a contingency basis in a closed loop system. Moreover, treatment therapy delivered can be immediate or delayed, diurnal, constant or intermittent depending on contingencies as defined by the closed loop system.

Referring now to FIG. 4, a schematic view of a display of an image analyzer providing multiple views of a patient's brain and multiple proposed trajectories for stimulation lead placement is illustrated, consistent with the present inventive concepts. Image analyzer 500 comprises user interface 501 and display 510. Shown on display 510 are multiple brain images. Also shown are a single target volume TV, and multiple avoidance volumes AV1, AV2 and AV3. AV1 and AV2 can comprise blood vessels of the brain. AV3 can comprise the optical tract or the fornix. Image analyzer 500 has provided multiple proposed trajectories, including trajectories TRA and TRB. Trajectories TRA and TRB can be identified based on having the all portions of each trajectory positioned away from avoidance volume AV1 and AV2 by at least a minimum distance. For example, AV1 and/or AV2 can comprise a blood vessel or a side wall of a ventricle of the brain (e.g. to avoid the stimulation lead sciving or deflecting off of the ventricle of the brain).

Trajectories TRA and TRB can be identified based on positioning the distal portion (e.g. a distal portion comprising one or more simulation elements) away from avoidance volume AV3, such as when the target volume TV comprises the anterior pillars of the fornix and the avoidance volume AV3 comprises the optical tract of the brain (e.g. to avoid subsequent stimulation of the optical tract). In some embodiments, the target volume TV comprises the fornix, and the avoidance volume AV3 also comprises the fornix, such as to identify a trajectory which positions a stimulation element proximate the fornix while preventing any portion of the stimulation lead from penetrating the fornix.

In some embodiments, trajectories TRA and TRB are identified based on multiple factors, such as two or more of: positioning one or more stimulation elements to maximize or otherwise achieve sufficient stimulation of target volume TV; positioning one or more stimulation elements to prevent or at least reduce stimulation of an avoidance volume such as avoidance volume AV3 such as when avoidance volume AV3 comprises the optical tract; positioning the stimulation lead to not penetrate avoidance volume AV3, such as when avoidance volume AV3 comprises the fornix and/or the optical tract; and positioning the stimulation lead to be a minimum distance from avoidance volume AV1 and/or AV2 along the entire length of the stimulation lead.

TRV1 comprises a cone shaped volume of tissue within which a large number of acceptable trajectories (e.g. TRA and TRV) can be selected for placement of one or more stimulation leads. One or more specific trajectories can be selected (e.g. TRA and/or TRB) and the system of the present invention can be configured to provide an quantitative or qualitative assessment of the trajectory selected, such as a quantitative or qualitative assessment based on effectiveness of stimulation of one or more target volumes TV and/or avoidance of one or more avoidance volumes AV. For example, as shown on display 510, trajectory TRA has been assessed quantitatively (e.g. via image analyzer 500 of FIG. 1) to be a 5 (e.g. 5 out of 10), and trajectory TRB has been assessed quantitatively to be a 7 (e.g. 7 out of 10), such as to suggest TRB would better stimulate one or more target volumes TV and/or better avoid one or more avoidance volumes AV.

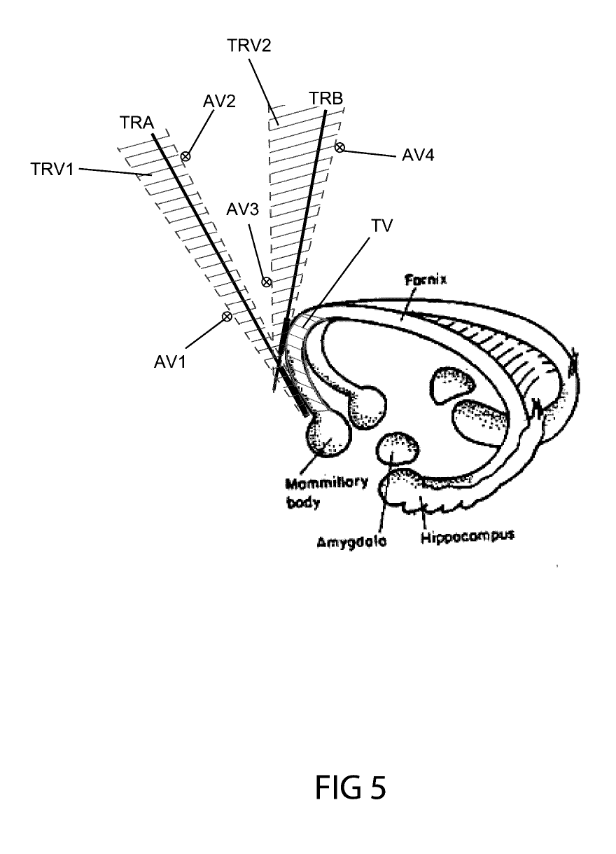

Referring now to FIG. 5, an anatomical view of a portion of a patient's brain is illustrated, including multiple identified trajectories for stimulation lead placement, consistent with the present inventive concepts. The image shown in FIG. 5 can be displayed on display 510 of image analyzer 500 described hereabove. A target volume TV has been selected comprising an anterior pillar of the fornix (e.g. by a clinician using image analyzer 500). Avoidance volumes AV1, AV2, AV3 and AV4 have been identified (e.g. be a clinician using image analyzer 500 and/or automatically by image analyzer 500). Image analyzer 500 has identified two volumes of acceptable trajectories, TRV1 and TRV2. TRV1 is selected to be between avoidance volumes AV1 and AV2 while allowing a distal portion of the trajectory to be positioned proximate a portion of target volume TV. TRV1 comprises trajectory TRA. TRV2 is selected to be between avoidance volumes AV3 and AV4 while allowing a distal portion of the trajectory to be positioned proximate a different portion of target volume TV. TRV2 comprises trajectory TRB.

TRA, TRB and/or another trajectory of TRV1 or TRV2 can be assessed (e.g. quantitatively or qualitatively assessed), such as by image analyzer 500 as described herein.

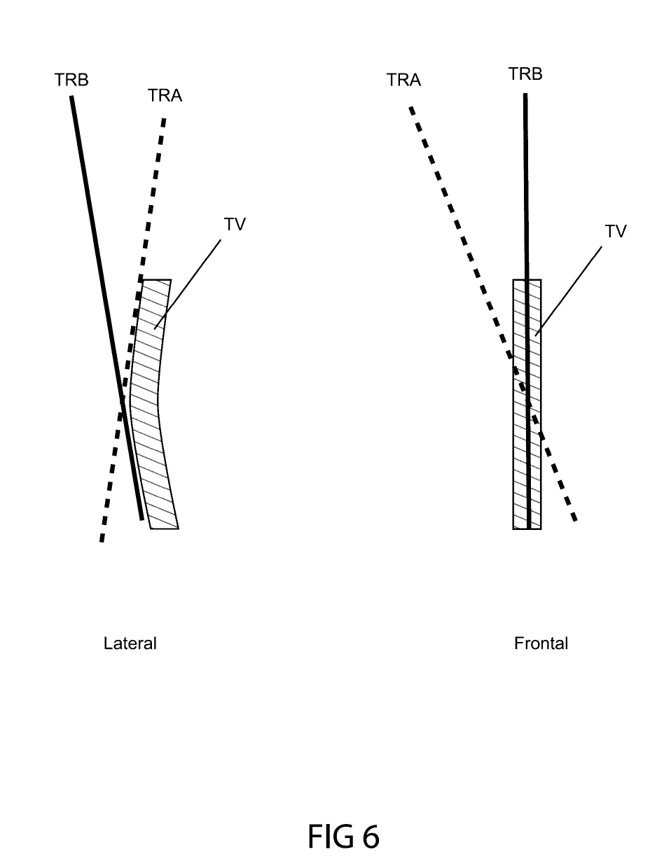

Referring now to FIG. 6, lateral and frontal anatomical views of a target volume of tissue to be stimulated are illustrated, consistent with the present inventive concepts. Target volume TV comprises a curved portion as shown in the lateral view. The two trajectories shown in each view, TRA and TRB can be compared. In the lateral view, each trajectory could appear to be in relatively close proximity to approximately half the length of target volume TV. However, in the frontal view, it is clear that trajectory TRA is in close proximity to target volume TV for only a very short portion of target volume TV. Image analyzer 500 would be configured to associate a more favorable quantitative and/or qualitative stimulation rating to trajectory TRB over trajectory TRA.

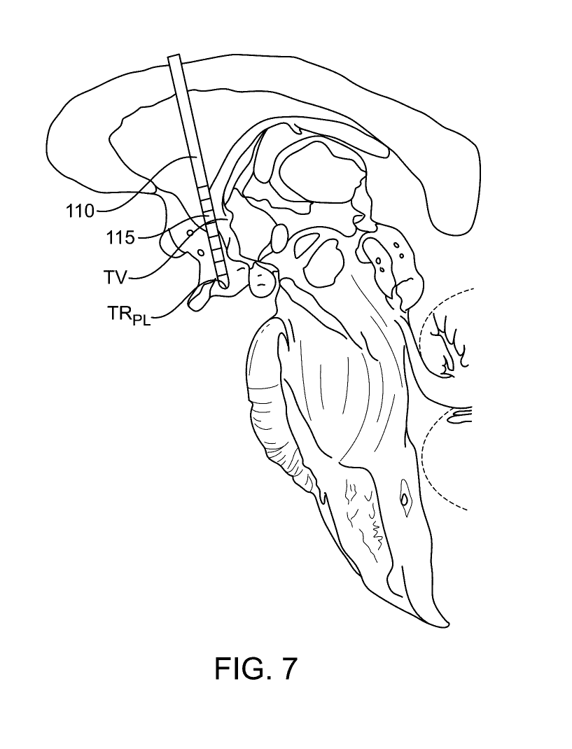

Referring now to FIG. 7, an anatomical view of a portion of a patient's brain with a distal portion of a stimulation lead implanted to stimulate the fornix is illustrated, consistent with the present inventive concepts. Stimulation lead 110 has been implanted along placement trajectory TR_{PL} such that its four stimulation elements 115 are positioned proximate to target volume TV, in this instance the fornix, such as to adequately stimulate the fornix when operatively attached to a stimulator as described herein. Placement trajectory TR_{PL} may have been identified and/or selected (e.g. via image analyzer 500 and/or an operator of image analyzer 500) to avoid one or more avoidance volumes AV, not shown but such as an avoidance volume selected from the group consisting of: a blood vessel; a sulcus of the brain surface; a wall of the ventricle (e.g. to avoid sciving or deflection); the optical tract (e.g. to avoid stimulation of the optical tract); the fornix (e.g. to avoid penetration of the fornix); and combinations of these.

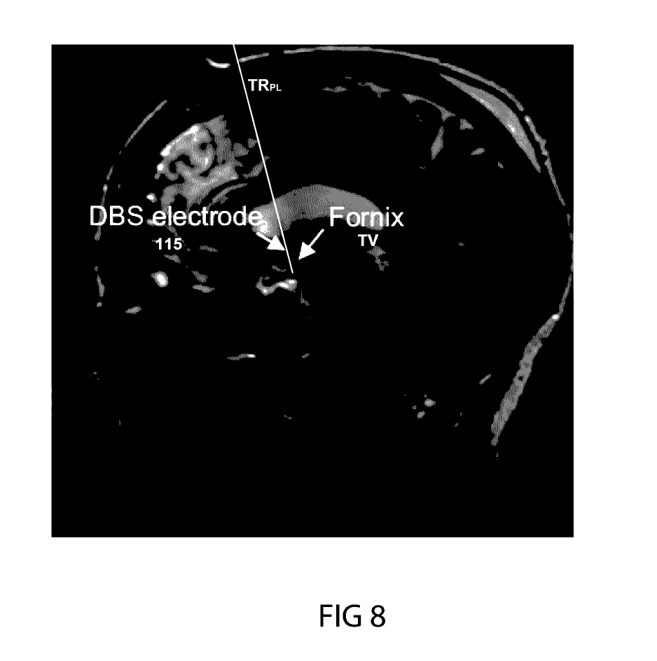

Referring now to FIG. 8, a side sectional image of a patient's brain with an implanted series of electrodes is illustrated, consistent with the present inventive concepts. Stimulation elements 115 are positioned along placement trajectory TR_{PL} such that a target volume TV comprising the fornix can be stimulated. Placement trajectory TR_{PL} can be identified and/or selected using system 10 and image analyzer 500 of the present inventive concepts.

The foregoing description and accompanying drawings set forth a number of examples of representative embodiments at the present time. Various modifications, additions and alternative designs will become apparent to those skilled in the art in light of the foregoing teachings without departing from the spirit hereof, or exceeding the scope hereof, which is indicated by the following claims rather than by the foregoing description. All changes and variations that fall within the meaning and range of equivalency of the claims are to be embraced within their scope.

What is claimed is:

1. A system for treating a cognitive disease or disorder comprising:
a stimulator configured to provide stimulation energy;
a first stimulation lead constructed and arranged to receive the stimulation energy from the stimulator and comprising at least one stimulation element constructed and arranged to stimulate a target volume of brain tissue;
a controller configured to modify the stimulation energy delivered by the stimulator;
an image analyzer configured to receive patient image information and identify at least one proposed trajectory for placement of the first stimulation lead, wherein the identification of the at least one proposed trajectory is based on:
the target volume of brain tissue, and
an avoidance volume of brain tissue;
wherein the system is configured to treat at least one of a neurological disease or a neurological disorder.

2. The system of claim 1, further comprising an imaging device configured to produce the patient image information.

3. The system of claim 2, wherein the imaging device comprises a device selected from the group consisting of: MRI; fMRI; X-ray; fluoroscope; Ct-Scanner; PET Scanner; Diffusion Tensor Imaging device; ultrasound imaging device; and combinations thereof.

4. The system of claim 1, further comprising a second stimulation lead constructed and arranged to receive the stimulation energy from the stimulator and comprising at least one stimulation element constructed and arranged to stimulate a second target volume of brain tissue.

5. The system of claim 4, wherein the target volume of brain tissue and the second volume of brain tissue comprises similar tissue.

6. The system of claim 1, wherein the stimulator is configured to provide energy selected from the group consisting of: electrical energy; magnetic field energy; light energy; optogenetic energy; sound energy; chemical energy; and combinations thereof.

7. The system of claim 1, wherein the first stimulation lead at least one stimulation element comprises a stimulation element selected from the group consisting of: an electrode such as one or more electrodes configured to deliver electrical stimulation energy; a magnetic field delivery element; a light delivery element such as a visible, ultraviolet or infrared light delivery element; an optogenetic delivery element; a sound delivery element such as a subsonic wave or ultrasound wave delivery element; an agent delivery element such as a chemical or pharmaceutical agent delivery element; and combinations thereof.

8. The system of claim 1, wherein the image analyzer device is configured to provide a qualitative measure of a selected trajectory.

9. The system of claim 8, wherein the qualitative measure comprises a qualitative measure of the at least on trajectory's avoidance of the avoidance volume.

10. The system of claim 9, wherein the qualitative measure is a qualitative measure of one or more of: proximity of the first stimulation lead to the avoidance volume and average distance between the first stimulation lead and the avoidance volume.

11. The system of claim 10, wherein the qualitative measure comprises a qualitative measure of the at least on trajectory's geometric relationship to the target volume.

12. The system of claim 11, wherein the qualitative measure is a qualitative measure of one or more of: proximity of a stimulation element to the target volume; average proximity of an array of stimulation elements to the target volume; and degree of parallelism of an array of stimulation elements to the target volume.

13. The system of claim 10, wherein the image analyzer device is configured to provide a first qualitative measure of a first trajectory and a second qualitative measure of a second trajectory.

14. The system of claim 1, wherein the image analyzer device is configured to provide a quantitative measure of a selected trajectory.

15. The system of claim 14, wherein the quantitative measure comprises a quantitative measure of the at least on trajectory's avoidance of the avoidance volume.

16. The system of claim 15, wherein the quantitative measure is a quantitative measure of one or more of: proximity of the first stimulation lead to the avoidance volume and average distance between the first stimulation lead and the avoidance volume.

17. The system of claim 14, wherein the quantitative measure comprises a quantitative measure of the at least on trajectory's geometric relationship to the target volume.

18. The system of claim 17, wherein the quantitative measure is a quantitative measure of one or more of: proximity of a stimulation element to the target volume; average proximity of an array of stimulation elements to the target volume; and degree of parallelism of an array of stimulation elements to the target volume.

19. The system of claim 14, wherein the image analyzer device is configured to provide a first quantitative measure of a first trajectory and a second quantitative measure of a second trajectory.

20. The system of claim 1, wherein the image analyer device is configured to provide information related to the distance between the first stimulation lead and the avoidance volume.

21. The system of claim 1, wherein the image analyzer device is configured to provide information related to the distance between the first at least one stimulation element at the target volume.

22. The system of claim 1, wherein the image analyzer device is configured to allow an operator to perform a function selected from the group consisting of: select a patient image; rotate an image; magnify a patient image; select a target volume; magnify the view of a target volume; view an avoidance volume; magnify an avoidance volume; view one or more stimulation lead trajectories identified by the image analyzer; analyze one or more stimulation lead trajectories identified by the image analyzer; select one or more stimulation lead trajectories identified by the image analyzer; measure the distance between one or more stimulation lead trajectories and a target volume; measure the distance between one or more stimulation lead trajectories and an avoidance volume; measure the distance between one or more stimulation elements proposed locations and a target volume; measure the distance between one or more stimulation elements proposed locations and an avoidance volume; average one or more measured distances; sum two or more measured distances; and combinations thereof.

23. The system of claim 1, wherein the image analyzer comprises the controller.

24. The system of claim 1, wherein the system further comprises a sensor.

25. The system of claim 24, wherein the sensor comprises a sensor selected from the group consisting of: neuronal activity sensor; EEG sensor; local field potential sensor; neurochemical sensor; pH sensor; pressure sensor; blood pressure sensor; optical sensor; blood gas sensor; blood oxygen sensor; magnetic sensor; strain gauge; temperature sensor; and combinations thereof.
